SEQUENCE LISTING (22 sequences)

SEQ ID NO: 1 - DNA, 21 bp, Homo sapiens
tcgacaactccgagtttcagc

SEQ ID NO: 2 - DNA, 39 bp, Homo sapiens
ctcctcgcggccgcatcgatccatagagcccaccgcatc

SEQ ID NO: 3 - DNA, 42 bp, herpes simplex virus 1
ctcctcaagcttctcgagcacacggagcgcggctgccgacac

SEQ ID NO: 4 - DNA, 35 bp, Herpes simplex virus 1
ctcctcggtaccccatggaggccagcagagccagc

SEQ ID NO: 5 - DNA, 42 bp, Herpes simplex virus 1
ctcctcgcggccgcactagttccgcgtgtccctttccgatgc

SEQ ID NO: 6 - DNA, 58 bp, Herpes simplex virus 1
ctcctcctcgagaagcttatgcatgagctcgacgtctcggcggtaatgagatacgagc

SEQ ID NO: 7 - DNA, 32 bp, Herpes simplex virus 1
ctcctcagaacccaggaccagggccacgttgg

SEQ ID NO: 8 - DNA, 33 bp, Herpes simplex virus 1
ctcctcatggagacaaagcccaagacggcaacc

SEQ ID NO: 9 - DNA, 36 bp, Herpes simplex virus 1
ctcctcggagaccggggttggggaatgaatccctcc

SEQ ID NO: 10 - DNA, 35 bp, Herpes simplex virus 1
ctcctcgcggggcgtgggaggggctggggcggacc

SEQ ID NO: 11 - DNA, 22 bp, Herpes simplex virus 1
agagggacatccaggactttgt

SEQ ID NO: 12 - DNA, 19 bp, Herpes simplex virus 1
caggcgcttgttggtgtac

SEQ ID NO: 13 - DNA, 16 bp, Herpes simplex virus 1
accgccgaactgagca

SEQ ID NO: 14 - DNA, 15 bp, Herpes simplex virus 1
cacgggccgcttcac

SEQ ID NO: 15 - DNA, 17 bp, Herpes simplex virus 1
gcgatagcgcgcgtaga

SEQ ID NO: 16 - DNA, 14 bp, Herpes simplex virus 1
ccgacgcgacctcc

SEQ ID NO: 17 - DNA, 17 bp, Herpes simplex virus 1
cctccacgcccaaaagc

SEQ ID NO: 18 - DNA, 22 bp, Herpes simplex virus 1
ggtggtgttgttcttgggtttg

SEQ ID NO: 19 - DNA, 15 bp, Herpes simplex virus 1
ccccacgtccacccc

SEQ ID NO: 20 - DNA, 25 bp, mus musculus
ctcaagaaatctaacccctgactca

SEQ ID NO: 21 - DNA, 16 bp, Mus musculus
gcgggacaggctgaga

SEQ ID NO: 22 - DNA, 15 bp, Mus musculus
ccccacacacacctc

(19) United States
(12) Patent Application Publication
(10) Pub. No.: US 2021/0401968 A1
(43) Pub. Date: Dec. 30, 2021

(54) Localized activation of virus replicatio boosts herpesvirus-vectored vaccines

(71) Applicant: HSF Pharmaceuticals SA, La Tour-d-Peliz (CH)

(72) Inventor: Richard W Voellmy, La Tour-d-Peilz (CH)

(73) Assignee: HSF Pharmaceuticals SA, La Tour-d-Peilz (CH)

(21) Appl. No.: 16/974,123

(22) PCT Filed: Mar. 8, 2019
(86) PCT No.: PCT/EP2019/055870

§ 371 Date: Oct. 14, 2020

Related U.S. Application Data
(60) Provisional application No. 62/710,995, filed on Mar. 8, 2018.

(51) Int. Cl. A61K 39/145

(52) U.S. Cl. CPC A61K 39/145; A61K 2039/5254

(57) ABSTRACT

The present invention relates to a vaccine composition comprising an effective amount of a replication-competent controlled herpesvirus expressing an antigen of a pathogen other than a herpesvirus. Encompassed are uses in immunization and methods of immunization employing the vaccine compositions, wherein transient activation of the replication of the herpesvirus at the site of vaccine administration to a subject enhances systemic immune responses to the antigen.

### TECHNICAL FIELD

The present disclosure relates to certain replication-competent controlled herpesviruses and their utilization for immunization against diseases other than herpetic diseases.

### BACKGROUND OF THE INVENTION

Vaccination is most probably the most cost-effective medical intervention that has saved countless human lives during its history of more than two hundred years. Among its most spectacular successes count the eradication of smallpox as well as the virtual disappearance of diphtheria, tetanus and paralytic poliomyelitis. Andre, F. E. (2003) Vaccinology: past achievements, present roadblocks and future promises. Vaccine 21: 593-5. In addition, vaccination has controlled, in at least part of the world, yellow fever, pertussis, Haemophilus influenzae type b, measles, mumps, rubella, typhoid and rabies. Still, important infections remain unpreventable as well as incapable of being treated by a therapeutic vaccine. Moreover, effectiveness of a number of vaccines is less than would be desirable. A particularly important example of a disease for which there is insufficient vaccine protection is influenza/flu. Clearly, the creation of new vaccines has not become routine, and development of immunization agents against diseases such as the flu may require new approaches.

Influenza is characterized typically by sudden fever, sore throat, cough, headache, myalgia, chills, anorexia and fatigue. Bridges, C. B. et al. Inactivated influenza vaccines. In: Vaccines (Plotkin, S. A. et al., eds.) 5th edition. 2008. Saunders Elsevier; Belshe, R. B. et al. Influenza vaccine-live. In: Vaccines (Plotkin, S. A. et al., eds.) 5th edition. 2008. Saunders Elsevier. Influenza is a high morbidity but relatively low mortality disease. Seasonal attack rates are said to be typically between 5% and 20%. The death toll from complications of the illness is considerable. According to the WHO, the worldwide yearly death toll may lie between 250,000 and 500,000. Influenza viruses are enveloped and contain a segmented negative-sense RNA genome. The spherical viral particles have spikes consisting of hemagglutinin (HA) and neuraminidase (NA). HA is the major antigen against which the host antibody response is directed. Influenza A viruses are classified into subtypes based on the properties of their envelope proteins HA and NA. Presently, influenza viruses of subtypes H3N2 and H1N1 are predominantly circulating in humans. Among the B type viruses, Yamagata- and Victoria-like strains appear to be presently prevalent. Influenza type A also infects birds including poultry, pigs, horses, dogs and even sea mammals. All known HA and NA subtypes could be isolated from wild aquatic birds, which constitute a natural reservoir and a source of genes for pandemic A-type viruses. B type viruses are found more exclusively in humans, but have been documented in horses and seals. Because of the error-prone mode of replication and selection in the host, influenza A and B viruses undergo gradual antigenic change in their two surface antigens, the HA and NA proteins. This phenomenon known as antigenic drift necessitates continuous vigilance and yearly review/update of strains used for vaccine production. Pandemics result from antigenic shift, i.e., introduction into the human population of a novel influenza A virus.

Whole-virus inactivated influenza vaccines have been in use since 1945. Typically, vaccine viruses have been propagated in the allantoic cavities of embryonated hens' eggs. More recently, such vaccines also have been made from viruses amplified in mammalian cell lines. Since the 1970s, most inactivated vaccines are subvirus or split vaccines. Typical vaccines in use are trivalent, comprising HAs from H1N1 and H3N2 subtype influenza A strains and an influenza B strain (referred to as TIV). Live attenuated influenza virus vaccines (LAIV) were developed more recently. An intranasal vaccine was made based on temperature-sensitive and cold-adapted influenza virus A and B strains.

A recent systematic review and meta-analysis of vaccine efficacy and effectiveness data was published by Osterholm et al. (Efficacy and effectiveness of influenza vaccines: a systematic review and meta-analysis. Lancet Infect. Dis. 12: 36-44 (2012)). The analysis focused on studies carried out in the United States and published between 1967 and 2011. Studies were selected based on a set of criteria that were intended to ensure scientific rigor and, to the extent possible, exclude bias. All criteria were fulfilled by 17 randomized, controlled trials showing vaccine efficacy (95% Cl>0), of which trials 8 related to TIV and 9 to LAIV. The trials covered 24 influenza seasons and included almost 54,000 participants. Of the trials that revealed significant efficacy for TIV, 6 involved 18-64-year-old participants, one children aged 6-23 months and one included all age groups and reported a combined efficacy. The mean vaccine efficacy revealed by these trials was 62%. It is noted that none of the trials specifically tested vaccine effects in adults 65 years of age and older or in children aged 2-17. Of particular interest is a study on young children that was carried on over two seasons in both of which there was a good match between vaccine and circulating strains. Hoberman, A. et al. (2003) Effectiveness of inactivated influenza vaccine in preventing acute otitis media in young children: a randomized controlled trial. JAMA 290: 1608-16. Vaccine efficacy in the first season was 66% and in the second −7%. Regarding LAIV, mean efficacy from eight studies in children aged 6 months to 7 years was 78%. Osterholm, M. T. et al. (2012). Three studies on subjects aged 18-49 revealed no significant protection. One study in persons over 60 showed an overall efficacy of 42%, but efficacy in 60-69-year-olds seemed to be considerably lower than that in persons over 70. No qualifying study related to children aged 8-17 or adults between 50 and 59 years of age.

Nine of 14 observational studies that satisfied the inclusion criteria reported effectiveness of seasonal influenza vaccine. Osterholm, M. T. et al. (2012). These studies included 17 embedded or cohort analyses. Six of the 17 analyses (35%) showed significant effectiveness against medically attended, laboratory-confirmed influenza. In children of 6-59 months, significant vaccine effectiveness was found in 3 of 8 seasons (38%). One of two such studies reported vaccine effectiveness in subjects aged 65 and older.

Based on the latter data it can be concluded that currently available, seasonally updated influenza vaccines provide moderate protection against virologically confirmed disease, which protection may not be long-lasting. No protection may be obtained in some seasons. Evidence for protection of the highest risk population, i.e., persons 65 years of age or over, is very thin indeed. More effective and non-seasonal vaccines are clearly needed. Present vaccines rely largely on the induction of HA antibodies for protective effects. It has been proposed that future influenza vaccines should be capable of inducing potent (effector) T cell responses, i.e., should induce more complete immune responses. Osterhaus, A. et al. (2011) Towards universal influenza vaccines. Phil. Trans. R. Soc. B 366: 2766-73; Thomas, P. G. et al. (2006) Cell-mediated protection in influenza infection. Emerging Infectious Diseases 12: 48-54.

In particular embodiments, the present disclosure relates to methods of cross-protective immunization against influenza infection or disease utilizing compositions comprising a replication-competent controlled herpesvirus or uses in cross-protective immunization of the latter compositions.

Replication-competent herpesviruses and virus pairs controlled by a SafeSwitch or a SafeSwitch-like gene switch were disclosed generally in U.S. Pat. Nos. 7,906,312 and 8,137,947, and international patent publication WO2016/030392.

### SUMMARY OF THE INVENTION

The present disclosure relates to the discovery that local presentation of an antigen of a pathogen other than a herpesvirus results in dramatically enhanced systemic immune responses to the latter antigen when this presentation occurs in the context of vigorous replication of a herpesvirus in the same locale. Presentation of the antigen of the pathogen as well as highly efficient viral replication at the site of vaccine administration (inoculation site) are provided by a herpesvirus-vectored vaccine that is a replication-competent controlled herpesvirus carrying an expressible gene for the antigen of the pathogen. A replication-competent controlled herpesvirus of the present disclosure is characterized as a recombinant herpesvirus in which one or more replication-essential genes have been placed under the control of a gene switch that is inserted in the genome of the recombinant herpesvirus and which replicates with an efficiency comparable to that of the wildtype herpesvirus from which it was derived when activated deliberately but essentially does not replicate when not activated. Highly efficient replication of a pathogenic virus is potentially dangerous. The replication-competent controlled herpesviruses of the present disclosure harbor a mechanism for the spatial and temporal control of their replication. Hence, their replication can be limited to occur only in a narrowly defined body region where the virus does not represent a danger. The time during which efficient replication occurs (or the number of cycles allowed) can also be stringently controlled. Ideally, replication triggered by one transient activating or activation treatment is limited to one cycle/round.

Hence, the present disclosure relates to the finding that presentation of an antigen of a pathogen (other than a herpesvirus) in the context of vigorous herpesvirus replication in a defined region of a subject's body (i.e., the region in which the replication-competent controlled herpesvirus has been administered, also called “inoculation site”) results in the induction of potent humoral and cellular immune responses against the antigen of the pathogen. Replication of known attenuated vaccine vectors is not limited to a particular body region. Hence, the attenuated replication of such vaccine vectors as well as the presentation of the antigens expressed by them occur in a disseminated fashion. Therefore, it was unknown whether a vaccine that presents a heterologous antigen in the context of vigorous, but transient virus replication only at the site of administration could, in fact, produce a significant enhancement of systemic immune responses to the antigen. Applicant's experimentation provides the answer to this question, at least for herpesvirus-vectored vaccines.

The present disclosure also relates to the use of a vaccine composition comprising an effective amount of a (one or more) herpesvirus-vectored vaccine, i.e., a (one or more) replication-competent controlled herpesvirus carrying an (one or more) expressible gene for an (one or more) antigen of a pathogen other than a herpesvirus for immunizing a subject with the aim of preventing infection of the subject by the pathogen or of reducing the effects of an infection by the pathogen in an infected subject. The use is characterized in that the humoral and cellular responses induced in the subject against the expressed antigen (one or more) of the pathogen are significantly enhanced by a transient activation of the replication of the (one or more) replication-competent controlled herpesvirus in the inoculation site. Alternatively, the method is characterized in that the humoral and cellular responses induced in the subject against the expressed antigen of the pathogen are significantly enhanced by a transient activation of the replication-competent controlled herpesvirus in the inoculation site, which activation causes the virus to undergo a single round of replication in the subject. As used herein, terms such as “significant enhancement”, “significantly enhanced” or “significantly enhances” relate to an improvement or increase in an immune response (a humoral or cellular response assessed by biochemical or immunological methods, or a protective response assessed in an appropriate animal model or in human subjects) caused by the localized, transient activation of a herpesvirus-vectored vaccine, which improvement or increase is significant based on a statistical analysis using criteria commonly used in the art.

Activated replication-competent controlled herpesvirus replicates with a comparable efficiency as the wildtype virus from which it has been derived. Efficiency is compared in a single-step growth experiment in a permissive cell line. Parallel cultures are infected with wildtype virus or replication-competent controlled herpesvirus at similar multiplicities of infection. Subsequent to activation of the replication-competent controlled herpesvirus (“activated” replication-competent controlled herpesvirus), the cultures are incubated for the time required for the completion of one round of replication by the wildtype virus. Efficiency is determined by plaque assay of the virus present in the compared cultures. Efficiencies are considered to be comparable if the compared cultures contain similar numbers of plaque-forming units (pfu). Numbers of pfu are considered to be similar if they differ by less than 10-fold. Replication-competent controlled herpesvirus is essentially non-replicating in the absence of activation (“unactivated” replication-competent controlled herpesvirus). The term “essentially non-replicating” means that in a single-step growth experiment, replication efficacy is a least 100-fold and, more preferably, at least 1,000-fold lower than that of activated replication-competent controlled herpesvirus or of the wildtype virus from which the replication-competent controlled herpesvirus has been derived. Thus, in practical terms, the unactivated replication-competent controlled herpesvirus is equivalent to a replication-deficient herpesvirus (obtained from mutagenesis or passage) or an inactivated (killed) herpesvirus.

The replication-competent controlled herpesvirus of the present disclosure that carries an expressible gene for an antigen of a pathogen has the following general properties:

(1) upon administration to a body region of a subject (the inoculation site), the replication-competent controlled virus remains essentially non-replicating in the absence of activation,

(2) exposure of the body region to a localized activation treatment activates the replication-competent controlled virus to undergo at least one round of replication in the body region, and

(3) upon localized activation, the replication-competent controlled virus replicates with an efficiency that is comparable to that of the wildtype virus from which it was derived and induces in the subject significantly more potent systemic immune responses to the antigen of the pathogen than those induced by the unactivated replication-competent controlled virus. The immune responses compared can be either antibody responses including neutralizing antibody responses, cellular immune responses such as antigen-specific T cell responses or protective responses against infection or disease caused by the pathogen in appropriate challenge models. The protective responses can relate to reducing infection or disease severity, disease duration or mortality subsequent to infection with said pathogen.

The present disclosure relates to uses for immunizing a subject with the aim of preventing infection by a pathogen or reducing the effects of an infection by the pathogen. These uses involve the administration to a subject of a herpesvirus-vectored vaccine composition comprising an effective amount of a (one or more) replication-competent controlled herpesvirus carrying an (one or more) expressible gene for an (one or more) antigen of a pathogen, whereby a transient activation of the (one or more) replication-competent controlled herpesvirus in the region in which it has been administered to the subject significantly enhances the systemic humoral and cellular responses induced in the subject against the antigen of the pathogen. In particular embodiments, replication of the replication-competent controlled herpesvirus is controlled by a gene switch that is activated by administration of a replication-activating (also simply referred to as “activating”) heat dose to the site of administration of the replication-competent controlled herpesvirus in the presence of an enabling concentration of a small-molecule regulator at the site of administration. In these embodiments, the replication-competent controlled (recombinant) herpesvirus is a heat- and small-molecule regulator-activated herpesvirus. Hence, the herpesvirus-vectored vaccine is a heat- and small-molecule regulator-activated herpesvirus carrying an expressible gene for an antigen of a pathogen that is not a herpesvirus.

The genome of a heat- and small-molecule regulator-activated herpesvirus comprises, as a consequence of insertion of or replacement in a genome of a wildtype herpesvirus of viral elements by heterologous elements that were introduced into the genome by any suitable means such as in vivo homologous recombination, a gene for a small-molecule regulator-activated transactivator which gene is functionally linked to a nucleic acid sequence that acts as a heat shock promoter or to a nucleic acid sequence that acts as a heat shock promoter as well as a transactivator-responsive promoter, and one or more transactivator-responsive promoters that are functionally linked to one or more viral genes that are required for efficient replication, which genes are also referred to as replication-essential genes. Subsequent to administration of the virus to a body region of a subject, it can be activated by subjecting the body region to an activating (replication-activating) heat dose in the presence of an effective (enabling) concentration in the body region of an appropriate small-molecule regulator. Such an activation treatment is expected to result in one round of virus replication. The replication-competent controlled herpesvirus further comprises an expressible gene or gene fragment encoding an antigen of a pathogen (other than a herpesvirus). Such gene or gene fragment can be expressed under the control of a constitutively active cellular or viral promoter or a transactivator-responsive promoter.

Alternatively, the genome of a heat- and small-molecule regulator-activated herpesvirus comprises, as a consequence of insertion of or replacement in a genome of a wildtype herpesvirus of viral elements by heterologous elements that were introduced into the genome by any suitable means such as in vivo homologous recombination, a gene for a small-molecule regulator-activated transactivator, which gene is functionally linked to a nucleic acid sequence that acts as a constitutively active or a transactivator-enhanced promoter, a nucleic acid sequence that acts as a heat shock promoter that is functionally linked to a first replication-essential viral gene, and a transactivator-responsive promoter that is functionally linked to a second replication-essential viral gene. Subsequent to administration of the virus to a body region of a mammalian subject, it can be activated by subjecting the body region to an activating heat dose in the presence of an effective concentration in the body region of an appropriate small-molecule regulator. Such an activation treatment is expected to result in one round of virus replication. The replication-competent controlled herpesvirus further comprises an expressible gene or gene fragment encoding an antigen of a pathogen (other than a herpesvirus). The term “transactivator-enhanced promoter” refers to a promoter that of necessity has some residual activity in the absence of transactivator and whose activity increases as a function of increasing levels of activated transactivator (also termed an auto-activated promoter). A “transactivator-responsive promoter” is a promoter that ideally is inactive in the absence of the transactivator that activates it. In uses in which the herpesvirus-vectored vaccine is a heat- and small-molecule regulator-activated herpesvirus transient activation consists of an application of a replication-activating heat dose to the site of inoculation of the vaccine in a subject in the presence of an enabling concentration of small-molecule regulator in said site.

In other, less preferred, embodiments, the vaccine composition comprises a replication-competent controlled herpesvirus whose genome has been made to comprise a gene for a small-molecule regulator-activated transactivator which gene is functionally linked to a nucleic acid sequence that acts as a constitutively active or a transactivator-enhanced promoter and a transactivator-responsive promoter that is functionally linked to a replication-essential viral gene. Subsequent to administration of the virus to a body region of a subject, it can be activated by subjecting the body region in a directed fashion to an effective concentration of an appropriate small-molecule regulator. Such an activation treatment results in at least one round of virus replication in the body region. The replication-competent controlled herpesvirus further comprises an expressible gene or gene fragment encoding an antigen of a pathogen (other than a herpesvirus). For convenience, the latter replication-competent controlled viruses are referred to herein as small-molecule regulator-activated herpesviruses.

In more specific (preferred) embodiments, the genome of the heat- and small-molecule regulator-activated herpesvirus comprises a gene for a small-molecule regulator-activated transactivator, which gene is functionally linked to a nucleic acid sequence that acts as a heat shock promoter or to a nucleic acid sequence that acts as a heat shock promoter as well as a transactivator-responsive promoter, and one or more transactivator-responsive promoters that are functionally linked to one or more replication-essential viral genes wherein one of the replication-essential viral genes is the ICP4 gene (both copies) or the ICP8 gene or two of the replication-essential viral genes are the ICP4 and ICP8 genes if the replication-competent controlled herpesvirus is derived from an HSV-1 or HSV-2, or functional analogs or orthologs of these genes if the replication-competent controlled herpesvirus is derived from another herpesvirus. The replication-competent controlled herpesvirus further comprises an expressible gene or gene fragment encoding an antigen of a pathogen (other than a herpesvirus).

In other more specific (preferred) embodiments, the genome of the heat- and small-molecule regulator-activated herpesvirus comprises a gene for a small-molecule regulator-activated transactivator which gene is functionally linked to a nucleic acid sequence that acts as a constitutively active or a transactivator-enhanced promoter, a nucleic acid sequence that acts as a heat shock promoter that is functionally linked to a first replication-essential viral gene and a transactivator-responsive promoter that is functionally linked to a second replication-essential viral gene wherein the second replication-essential viral genes is the ICP4 gene if the replication-competent controlled herpesvirus is derived from an HSV-1 or HSV-2, or a functional analog or ortholog of this gene if the replication-competent controlled herpesvirus is derived from another herpesvirus. The replication-competent controlled herpesvirus further comprises an expressible gene or gene fragment encoding an antigen of a pathogen (other than a herpesvirus).

The herpesvirus-vectored vaccine, i.e., the replication-competent controlled virus comprising an expressible gene or gene fragment encoding an antigen of a pathogen (other than a herpesvirus), can be derived from a virus of the herpesviridae family. In more specific embodiments, it is derived from a virus selected from an HSV-1, an HSV-2, a varicella zoster virus and a cytomegalovirus.

In more specific uses of a herpesvirus-vectored vaccine or vaccine composition, the replication-competent controlled herpesvirus comprising an expressible gene or gene fragment encoding an antigen of a pathogen (other than a herpesvirus) is a heat- and small-molecule regulator-activated virus, whereby the virus is derived from an HSV-1 or HSV-2 and a transactivator-controlled replication-essential viral gene is all copies of the ICP4 gene or the ICP8 gene. More specifically, the virus is derived from HSV-GS1. Alternatively, the virus is derived from an HSV-1 or HSV-2 and a first transactivator-controlled replication-essential viral gene is all copies of the ICP4 gene and a second transactivator-controlled or heat shock promoter-driven replication-essential viral gene is the ICP8 gene. More specifically, the virus is derived from HSV-GS3. In other specific embodiments, the heat- and small-molecule regulator-activated virus or small-molecule regulator-activated virus is derived from an HSV-1 or HSV-2 and lacks a functional ICP47 gene. This virus can be derived from HSV-GS4.

In a preferred heat- and small-molecule regulator-activated herpesvirus or small-molecule regulator-activated herpesvirus of the present disclosure, the small-molecule regulator-activated transactivator contains a ligand-binding domain from a progesterone receptor and is activated by a progesterone receptor antagonist (antiprogestin) or other molecule capable of interacting with the ligand-binding domain and of activating the transactivator. Transient activation of a replication-competent controlled herpesvirus that employs such a transactivator consists of an application in a subject of a replication-activating heat dose to the body region in which the herpesvirus has been administered in the presence of an enabling concentration of an antiprogestin in said body region. Specific preferred antiprogestins are ulipristal and mifepristone. Alternatively, the transactivator can contain a ligand-binding domain from an ecdysone receptor and is activated by an ecdysteroid, a diacylhydrazine or other molecule capable of interacting with the ligand-binding domain and of activating the transactivator. In yet another alternative embodiment, it contains a ligand-binding domain from a bacterial tetracycline repressor and is activated by a tetracycline or other molecule capable of interacting with the tetracycline repressor domain and of activating the transactivator. In yet another alternative embodiment, the small-molecule regulator-activated transactivator contains a ligand-binding domain from an estrogen receptor and is activated by an estrogen receptor antagonist or other molecule capable of interacting with the ligand-binding domain and of activating the transactivator. In a further embodiment, the small-molecule regulator-activated transactivator is a complex of a polypeptide containing an FKBP12 sequence and a polypeptide containing an FRB sequence from mTOR, and is activated by rapamycin, a rapamycin derivative (rapalog) or other molecule capable of interacting with both polypeptides and of activating the transactivator.

It is noted that a composition for immunization, in which the active component is a heat- and small-molecule regulator-activated herpesvirus or a small-molecule regulator-activated herpesvirus, can further comprise an effective amount of a small-molecule regulator that is capable of activating the transactivator that controls the replication of the latter replication-competent controlled herpesvirus.

In the immunization uses described herein, the herpesvirus-vectored vaccine or composition comprising a replication-competent controlled herpesvirus can be administered to any region near the surface of or within the body of a subject to which region a replication-activating treatment, e.g., a heat dose and/or an effective dose of an appropriate small-molecule regulator, can be locally or regionally delivered. Preferably, the site of inoculation of the vaccine is a cutaneous or subcutaneous region located anywhere on the trunk or on an extremity of the subject. More preferably, administration of the composition is to a cutaneous or subcutaneous region located on an upper extremity of the subject. Administration can also be to a mucous membrane in an orifice of a subject, e.g., the nasal mucous membrane of a subject. In the case of a heat- and small-molecule regulator-activated herpesvirus, the activating heat dose can be administered by means of a heating pad that is applied to the cutaneous or subcutaneous site to which the replication-competent controlled herpesvirus has been administered.

Preferred embodiments relate to the use of a composition comprising effective amounts of one or more (kinds of) replication-competent controlled herpesviruses each carrying at least one expressible gene for an antigen of an influenza virus strain for immunization of a subject against influenza, wherein the replication-competent controlled herpesvirus is characterized as a recombinant herpesvirus in which one or more replication-essential genes have been placed under the control of a gene switch that is inserted in the genome of the recombinant herpesvirus and which replicates with an efficiency comparable to that of the wildtype herpesvirus from which it was derived when activated deliberately but when not activated does not detectably replicate or, in the alternative, replicates with a more than 100-fold lower efficiency than the wildtype virus. Also encompassed is the use of a composition comprising an effective amount of a replication-competent controlled herpesvirus carrying an expressible gene for an antigen of an influenza virus strain for immunization of a subject against influenza, wherein immunization comprises administration of the composition to a desired inoculation site on the subject, and exposing the inoculation site to a transient activation treatment that triggers at least one round of local replication of the replication-competent controlled herpesvirus. The replication-competent controlled herpesvirus can be derived from a virus selected from the group consisting of an HSV-1, an HSV-2, a varicella zoster virus and a cytomegalovirus. More specifically, the gene switch contained in the replication-competent controlled herpesvirus can be a gene switch that is co-activated by heat and a small-molecule regulator. In the latter case, transient activation entails an application of a replication-activating heat dose to the site of administration in the presence of an enabling concentration of small-molecule regulator in said site. The small molecule regulator can be an antiprogestin, if the gene switch comprises a transactivator containing a truncated progesterone receptor ligand-binding domain. The replication-competent controlled herpesvirus can be a recombinant HSV-1 or HSV-2 and the replication-essential viral genes that are functionally linked to transactivator-responsive promoters include at least all copies of the ICP4 gene or the ICP8 gene. The inoculation site can be a cutaneous or subcutaneous region on the trunk or on an extremity of the subject.

The latter preferred embodiments relate to the use of a composition comprising effective amounts of one or more replication-competent controlled herpesviruses (each) carrying an expressible gene for an antigen of an influenza virus strain for immunization of a subject against influenza. The gene for the influenza antigen can originate from an influenza strain that currently circulates in the human population. Particularly preferred are uses for cross-protective immunization, wherein the influenza virus strain from which the antigen originates (or is derived) differs genetically from any influenza virus strain circulating at the time of immunization. The genetic difference manifests itself in the nucleotide sequences of antigen gene-providing virus and target virus of immunization. Relevant in this regard are nucleotide sequences of hemagglutinin (HA) and neuraminidase (NA) genes. The genetic difference can also manifest itself in a serologic difference. Hence, the influenza virus from which the antigen is derived will be heterologous with regard to the virus against which immunization is to be directed. The viruses may differ in clade or may even differ in HA or NA subtype. The term “influenza virus strain circulating at the time of immunization” refers to virus strains identified by competent regulatory bodies or institutions engaged by such regulatory bodies that are concerned with the identification of virus strains to be targeted by the next seasonal vaccine. Alternatively, the term can refer more generally to virus strains identified by medical institutions and/or regulatory agencies to be prevalent in the season concerned in an unvaccinated population (e.g., in a state, a country or a continent). In addition, or in the alternative, the influenza virus strain from which an antigen-encoding gene will be harnessed will typically be a historical strain, i.e., a strain that was prevalent in an earlier season.

The expressible gene for an antigen of an influenza virus strain can be a gene or gene fragment encoding any envelope protein or parts thereof, or a gene or gene fragment encoding any internal viral protein or parts thereof. More preferably, the expressible gene is a gene or gene fragment encoding all or part of a nucleoprotein, a hemagglutinin, a neuraminidase, an ion channel protein or a matrix protein. The expressible influenza gene can originate from a human type A or type B influenza virus. Expression of the influenza gene or gene fragment can be driven by constitutively active promoter. Such a promoter can be a cellular promoter or a viral promoter. Preferred is a viral promoter, in particular the CMV immediate-early gene promoter. The promoter may also be a transactivator-responsive promoter or a heat shock promoter.

Transient activation of efficient replication of a herpesvirus-vectored influenza vaccine in the site of its administration will significantly enhance systemic immune responses to the vaccine, without compromising vaccine safety. Encompassed in particular is the induction of significant immune responses against an influenza virus strain that is a different strain than the strain from which the antigen gene present in the vaccine was derived. Furthermore, also encompassed is the induction of significant immune responses against an influenza virus that is in a different clade than the influenza virus from which the antigen gene present in the vaccine was derived or against an influenza virus strain that differs in subtype (hemagglutinin/neuraminidase) from the influenza virus strain from which the antigen gene present in the vaccine was derived.

Encompassed in the uses and methods described herein are second and further transient activations of a herpesvirus-vectored vaccine or booster immunizations intended to further enhance immune responses.

### BRIEF DESCRIPTION OF FIGURES

FIG. 1 presents an antiprogestin (mifepristone)-armed and heat-activated (or heat- and antiprogestin (mifepristone)-activated) SafeSwitch controlling a luciferase target gene. Reproduced from Vilaboa, N. and Voellmy, R. (2009) Deliberate regulation of therapeutic transgenes. In: Gene and Cell Therapy: Therapeutic Mechanisms and Strategies, Third Edition (Smyth Templeton, N. ed.) CRC Press, Boca Raton, Fla., pp. 619-36.

FIG. 2 relates to SafeSwitch performance in a stably transfected cell human line. Top panel: target gene activity one day after activating heat treatment (HS) at 43° C. Bottom panel: gene activity 1 day (1 d) and 6 days (6 d) after heat activation (43° C./2 h) in the presence of mifepristone (Mif), and reversibility of activation. *Mif was washed away one day after HS. Reproduced from Vilaboa, N. and Voellmy, R. (2009).

FIG. 3 relates to single step growth experiments with HSV-GS1 in Vero cells. (A) Controllability of replication. Four basic conditions were tested: (1) heat treatment at 43.5° C. for 30 min in the presence of 10 nM mifepristone (activating treatment), (2) heat treatment alone, (3) mifepristone exposure alone, and (4) no treatment. Heat treatment was administered immediately after infection (i.e., immediately after removal of the viral inoculum). (B) Comparison of replication efficiencies of wild type strain 17syn+ and HSV-GS1 with or without activating treatment. Heat treatment was applied 4 h after infection. Mif: mifepristone. PFU/ml values and standard deviations are shown.

FIG. 4 relates to single step growth experiments with HSV-GS3. (A) Comparison of replication efficiencies of wild type strain 17syn+ and HSV-GS3 with or without activating treatment in E5 cells (17syn+) or E5 cells transfected with an ICP8 expression plasmid (HSV-GS3). (B) Regulation of replication of HSV-GS3 in Vero cells. See the legend to FIG. 3 for a description of the four basic conditions tested (C) Analogous experiment in SCC-15 cells. In these experiments, heat treatments were administered immediately after infection. PFU/ml values and standard deviations are shown.

FIG. 5 relates to regulation of viral DNA replication and transcription in Vero cells infected with HSV-GS3. Multiple infected cultures were subjected to the treatments indicated in the panels. Heat treatment (43.5° C. for 30 min) was administered 4 h after infection, and sets of cultures were harvested 1, 4, 12 and 24 h later, and DNA and RNA were extracted and analyzed by qPCR and RT-qPCR, respectively. Uli: ulipristal. (A) HSV DNA. (B) ICP4 RNA. (C) gC RNA. Values and standard deviations were normalized relative to the highest value in each panel.

FIG. 6 relates to regulation of HSV-GS3 DNA replication and transcription, and comparison of replicative yields between HSV-GS3 and KD6 in the mouse footpad model. Adult outbred mice were inoculated on the slightly abraded footpads of their hind legs with 1×10⁵ PFU of HSV-GS3 or KD6. Indicated doses of ulipristal were administered intraperitoneally at the time of infection. Localized heat treatment at 45° C. for 10 min was performed 3 h after virus administration. Mice were sacrificed 24 h (panels A-C) or 4 days (panel D) post heat treatment, and DNA and RNA were isolated from feet and dorsal root ganglia (DRG) and analyzed by qPCR and RT-qPCR, respectively. (A) HSV DNA. (B) ICP4 RNA. (C) gC RNA. (D) HSV DNA at 4 days post heat treatment. Values and standard deviations were normalized relative to the highest value in each panel. ND: none detected.

FIG. 7 relates to humoral and cellular immune responses to an equine influenza virus (EIV) hemagglutinin (HA) induced by immunization with HSV-GS11. Adult female mice were vaccinated on both rear footpads with either saline (mock), HSV-GS3, or HSV-GS11. Herpesvirus-vectored vaccines were activated in some treatment groups by administration of heat to the rear feet and ulipristal i.p. One treatment group received a second heat and ulipristal activation two days after the first activation. A. Detection of EIV HA RNA in feet 24 h after the last treatment. Data are presented in relative quantities. N=5 per group and ***=p≤0.05; B. Detection of EIV HA antigen in feet 24 h after the last treatment. ELISA data are presented in relative units of fluorescence. N=5 per group and ***=p≤0.05; C. Neutralizing antibodies induced twenty-one days post vaccination in serum samples. Values are presented as percent of EIV Prague/56 pfu neutralized for each experimental group. N=5 per group and ***=p≤0.05; D) EIV Prague/56 HA specific lymphocytes induced twenty-one days post vaccination determined by a limiting dilution lymphocyte proliferation assay. The data are presented as responder cell frequency of each experimental group. N=5 per group and ***=p≤0.05.

FIG. 8. A method for the activation of HSPA7/HSP70B and HSPA1A promoters in the human skin. (a) Photograph of a heating pad. (b) Photographs showing how a heating pad is placed and fastened to a forearm of a subject. (c) Temperature on the skin surface during a 15-min heat treatment. (d) RT-qPCR data for 3 subjects. HSPA1A and HSPA7 RNA quantities were normalized using cellular β2-microglobulin RNA and expressed as relative quantities. Multiple tissue sections for each tissue compartment were pooled, and RNA was extracted using an RNeasy Mini Kit (Qiagen) and reverse-transcribed employing the QuantiTect RT Kit (Qiagen). RNA was quantified using a NanoDrop spectrophotometer. cDNA was amplified using a SYBR-Green RT-PCR kit from Qiagen. (For B2M primers see Cicinnati V R, Shen Q, Sotiropoulos G C, Radtke A, Gerken G et al. (2008) Validation of putative reference genes for gene expression studies in human hepatocellular carcinoma using real-time quantitative RT-PCR. BMC Cancer 8:350; for HSPA1A and HSPA7 primers see Villa F, Carrizzo A, Spinelli C C, Ferrario A, Malovini A et al. (2015) Genetic analysis reveals a longevity-associated protein modulating endothelial function and angiogenesis. Circ Res 117:333-345.)

### DETAILED DESCRIPTION

Unless otherwise defined below or elsewhere in the present specification, all terms shall have their ordinary meaning in the relevant art.

“Replication of virus” or “virus/viral replication” are understood to mean multiplication of viral particles. Replication is measured by determination of numbers of infectious virus, e.g., plaque-forming units of virus (pfu).

“Proteotoxic stress” is a physical or chemical insult that results in increased protein unfolding, reduces maturation of newly synthesized polypeptides or causes synthesis of proteins that are unable to fold properly.

A “small-molecule regulator” is understood to be a low molecular weight ligand of a transactivator used in a replication-competent controlled virus. The small-molecule regulator is capable of activating the transactivator. The small-molecule regulator is typically, but not necessarily, smaller than about 1000 Dalton (1 kDa).

The term “transactivator” is used herein to refer to a non-viral and, typically, engineered transcription factor that when activated by a small-molecule regulator can positively affect transcription of a gene controlled by a transactivator-responsive promoter.

“Activated” when used in connection with a transactivated gene means that the rate of expression of the gene is measurably greater after activation than before activation. When used in connection with a transactivator, “active” or “activated” refers to a transactivation-competent form of the transactivator. When used in connection with a replication-competent controlled virus, the term means that replication of the virus has been triggered.

“Promoter of a heat shock gene”, “heat shock gene promoter” and “heat shock promoter” are used synonymously. A “nucleic acid that acts as a heat shock promoter” can be a heat shock promoter or a nucleic acid that contains sequence elements of the type present in heat shock promoters which elements confer heat activation on a functionally linked gene.

Herein, a virus, whose genome includes a foreign (heterologous) non-viral or viral gene (e.g., a gene for an antigen of a pathogen other than a herpesvirus), is either referred to as a “virus” or a “viral vector”.

A “wildtype virus” is a virus that has been isolated from a subject or the environment and, although propagated in vitro or in animals, has not been subjected to any deliberate selection or mutational process.

A “replication-competent controlled herpesvirus” is a recombinant herpesvirus whose replicative ability is under the control of a gene switch that can be deliberately activated.

A “recombinant (herpes)virus” refers specifically to a virus that has been altered by an experimenter. Often, a “recombinant (herpes)virus” is simply referred to as a “(herpes)virus” or as a “recombinant”.

A “replication-essential gene” or a “gene required for efficient replication” is arbitrarily defined herein as a viral gene whose loss of function diminishes replication efficiency by a factor of at least 10, preferably by a factor of a least 100. Replication efficiency is typically estimated in a single step growth experiment. For many viruses it is well known which genes are replication-essential genes. For herpesviruses see, e.g., Nishiyama, Y. (1996) Herpesvirus genes: molecular basis of viral replication and pathogenicity. Nagoya L. Med. Sci. 59: 107-19.

A “herpesvirus-vectored vaccine” refers to a replication-competent controlled herpesvirus that expresses an antigen of a pathogen other than a herpesvirus, i.e., whose genome contains an expressible gene for an antigen of a pathogen other than a herpesvirus.

“Transient activation” means that activation of virus replication is triggered by a single treatment, also referred to as “activation treatment”, that is aimed ideally at causing one round of replication of a replication-competent controlled herpesvirus or, less ideally, one or more rounds of replication over a period of at most several days. For a heat- and small-molecule regulator-activated herpesvirus of the present disclosure, the term would mean that an appropriate (replication-activating) heat dose is administered once to virus-infected cells in the presence of an enabling concentration of the appropriate small-molecule regulator.

“Site of administration” or “inoculation site” is a discrete region including and surrounding the point or area of application of a herpesvirus-vectored vaccine composition of this disclosure. The “site of administration” is intended to include the proximal, typically contiguous, tissue area in which initial localized infection by the vaccine virus occurs. In the model experiments described herein, the site of administration is the plantar surfaces of the hindfoot of a mouse. In a human subject, it may be a cutaneous or subcutaneous area on an extremity or the trunk that includes the point/area of vaccine application. More preferably, it may be a square or rounded area of about 100 cm² or smaller centered around the point/area of vaccine application. It may also be the mucous nasal, vaginal or anal membranes of a human subject.

“Transient activation . . . at the site of administration” means that activation of replication (of a herpesvirus-vectored vaccine) is achieved by focused administration of an activation treatment to the site of administration of the herpesvirus-vectored vaccine. For a heat- and small-molecule regulator-activated herpesvirus of the present disclosure, the expression would mean that an appropriate (replication-activating) heat dose is administered once to the site of administration of the vaccine in the presence in the site/area or systemically of an enabling concentration of the appropriate small-molecule regulator. If the heat dose is administered to an administration site on the trunk or an extremity of a human subject, heat may be applied to a region that is essentially co-extensive with the administration site as defined above. In the model experiments disclosed herein, the site of administration was the plantar surfaces of a mouse hindfoot, and localized heat treatment was administered by immersing the hindfoot in a temperature-controlled water bath.

An “effective amount” of a herpesvirus-vectored vaccine or a replication-competent controlled herpesvirus expressing an antigen of a pathogen other than a herpesvirus is an amount of such virus that upon administration to a subject followed by localized activation (typically, at the site of administration) detectably enhances a subject's immune responses to the expressed heterologous antigen including resistance to infection by the pathogen from which the antigen expressed by the virus has been derived, and/or detectable reduction of disease severity, disease duration or mortality subsequent to infection of the vaccinated subject with said pathogen.

An “effective amount of a small-molecule regulator” is an amount that when administered to a subject by a desired route results in an “enabling concentration” of the molecule which concentration is capable of co-activating (in combination with a heat treatment) a heat- and small-molecule regulator-activated virus or activating a small-molecule regulator-activated virus with which the subject concurrently is, has been or will be inoculated to undergo a round of replication (or at least one round of replication in the case of a small-molecule regulator-activated virus) in the administration region.

A “subject” is a mammalian animal or a human person.

A “heat shock gene” is defined herein as any gene, from any eukaryotic organism, whose activity is enhanced when the cell containing the gene is exposed to a temperature above its normal growth temperature. Typically, such genes are activated when the temperature to which the cell is normally exposed is raised by 3-10° C. Heat shock genes comprise genes for the “classical” heat shock proteins, i.e., HSP110, HSP90, HSP70, HSP60, HSP40, and HSP20-30. They also include other heat-inducible genes, including genes for MDR1, ubiquitin, FKBP52, heme oxygenase and other proteins. The promoters of these genes, the “heat shock promoters”, contain characteristic sequence elements referred to as heat shock elements (HSE) that consist of perfect or imperfect 5-bp-long sequence modules that are arranged in alternating orientations. Amin, J. et al. (1988) Key features of heat shock regulatory elements. Mol. Cell. Biol. 8: 3761-3769; Xiao, H. and Lis, J. T. (1988) Germline transformation used to define key features of heat-shock response elements. Science 239: 1139-1142; Fernandes, M. et al. (1994) Fine structure analyses of the Drosophila and Saccharomyces heat shock factor—heat shock element interactions. Nucleic Acids Res. 22: 167-173. These elements are highly conserved in all eukaryotic cells such that, e.g., a heat shock promoter from a fruit fly is functional and heat-regulated in a frog cell. Voellmy, R. and Rungger, D. (1982) Transcription of a Drosophila heat shock gene is heat-induced in Xenopus oocytes. Proc. Natl. Acad. Sci. USA 79: 1776-1780. HSE sequences are binding sites for heat shock transcription factors (HSFs; reviewed in Wu, C. (1995) Heat shock transcription factors: structure and regulation. Annu. Rev. Cell Dev. Biol. 11, 441-469). The transcription factor primarily responsible for activation of heat shock genes in vertebrate cells exposed to heat or a proteotoxic stress is heat shock transcription factor 1 (referred to herein as “HSF1”). Baler, R. et al. (1993) Activation of human heat shock genes is accompanied by oligomerization, modification, and rapid translocation of heat shock factor HSF1. Mol. Cell. Biol. 13: 2486-2496; McMillan, D. R. et al. (1998) Targeted disruption of heat shock factor 1 abolishes thermotolerance and protection against heat-inducible apoptosis. J. Biol. Chem. 273, 7523-7528. Preferred promoters for use in replication-competent controlled viruses discussed herein are those from inducible HSP70 genes. A particularly preferred heat shock promoter is the promoter of the human HSP70B gene. Voellmy, R. et al. (1985) Isolation and functional analysis of a human 70,000-dalton heat shock protein gene fragment. Proc. Natl. Acad. Sci. USA 82, 4949-4953.

As was alluded to under Background, current thought appears to be that in order to be effective or more effective, respectfully, improved vaccine candidates for preventing or treating diseases such as herpes, HIV, tuberculosis or influenza need to elicit a balanced immune response that also includes a powerful effector T cell response.

Applicant developed the notion that a heterologous antigen expressed from a herpesvirus would induce the strongest and most balanced immune responses if it were presented in the context of vigorous, unattenuated replication of the herpesvirus. Clearly, a use of a pathogenic virus such as a wildtype herpesvirus as a vaccine is no longer acceptable. (It was historically, when procedures such a variolation were practiced.) The next best thing would be to genetically modify a wildtype herpesvirus such that its replication can be deliberately controlled. When activated, the genetically modified herpesvirus should replicate with the same or a similar efficiency as the wildtype virus from which it was derived. Applicant has developed such genetically modified herpesviruses, referred to as replication-competent controlled herpesviruses, starting from a virulent HSV-1 wildtype virus. To achieve a safe immunization, disseminated replication with the attendant danger of causing, depending on the type of herpesvirus, encephalitis, blindness, genital lesions, widespread painful skin rashes, mononucleosis, hepatitis, etc., must be avoided to the maximal extent achievable. Replication must be limited in time and restricted to occur in a body region in which no disease can be triggered and a minor amount of tissue damage can be accepted. Therefore, replication-competent controlled herpesviruses are to be activated in defined locales, i.e., in the selected site of administration. Preferably, the viruses are administered to small areas on/near a surface of a subject's body, preferably cutaneously or subcutaneously on the trunk or an extremity of the subject.

To applicant's knowledge, the question whether a vaccine virus that is capable of replicating, in a controlled fashion, with the efficiency of a wildtype virus, at the site of administration can produce a significantly stronger and more balanced systemic immune response against a heterologous antigen expressed from the virus than a non-replicating vector expressing the same antigen had never been addressed. Therefore, the answer was not foreseeable. The limited research performed prior to applicant's work is not relevant to the question as it related to immune responses to heterologous antigens expressed from attenuated replication-competent virus vectors, hence to immune responses elicited by disseminated viruses that replicated weakly and in an uncontrolled fashion. Applicant approached the question using its replication-competent controlled herpesviruses as vectors for delivering influenza virus antigens. The present disclosure is based on applicant's unexpected finding that activation of the replication of replication-competent controlled herpesviruses in the region of their administration resulted in a dramatic enhancement of immune responses to influenza proteins expressed from the replication-competent controlled herpesviruses.

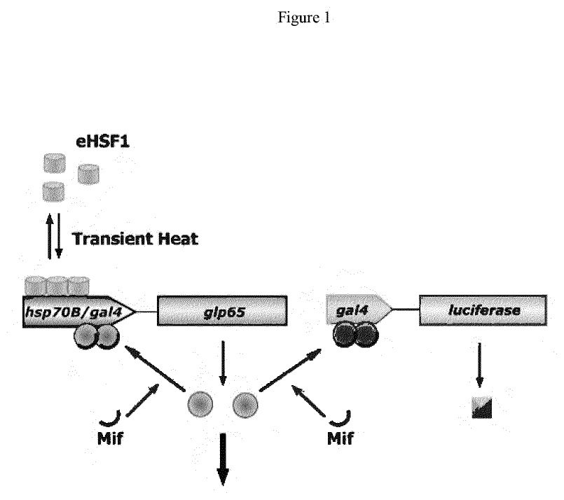

To generate a replication-competent controlled herpesvirus, a wild type virus is genetically altered by placing at least one selected replication-essential gene under the control of a gene switch that has a broad dynamic range, i.e., that essentially functions as an on/off switch. Most preferred are heat- and small-molecule regulator-activated (dual-responsive) gene switches that were discussed, e.g., in Vilaboa, N. et al. (2011) Gene switches for deliberate regulation of transgene expression: recent advances in system development and uses. J. Genet. Syndr. Gene Ther. 2: 107. A particular gene switch of this kind, referred to as SafeSwitch (co-activated by heat and an antiprogestin), has been used in Examples and is illustrated in FIG. 1. Unless specifically indicated, the description that follows relates to viruses whose replication has been brought under the control of such a dual-responsive gene switch. However, the description is also relevant to other replication-competent controlled viruses (e.g., other types of heat- and small-molecule regulator-activated viruses and small-molecule regulator-activated viruses).

Replication of the so modified virus, a heat- and small-molecule regulator-activated virus, only occurs when the dual-responsive gene switch is armed by an appropriate small-molecule regulator as well as is triggered by transient heat treatment (at a level below that causing burns or pain but above that which may be encountered in a feverish patient). Once the gene switch is activated, the virus expresses the full complement of viral proteins (or the desired complement of viral proteins) and replicates with an efficiency that is comparable to that of wild type virus (the virus from which the regulated virus was derived).

Heat can be readily focused. Administering focused heat to a body region to which a replication-competent controlled virus has been administered (i.e., the administration site) to trigger activation of the dual-responsive gene switch (in the presence of small-molecule regulator) will result in virus replication that is confined to the heated region. The dual requirement for heat and a small-molecule regulator is intended to provide a high level of security against accidental virus replication. In the absence of small-molecule regulator, activation/re-activation of virus is virtually impossible. Similarly, in the absence of a concomitant heat treatment, virus replication is not normally activated.

The arming small-molecule regulator needs to satisfy a number of criteria. Most important will be that the substance is safe; adverse effects should occur at most at an extremely low rate and should be generally of a mild nature. Ideally, the chosen small-molecule regulator will belong to a chemical group that is not used in human therapy. However, before any substance not otherwise developed for human therapy could be used as small-molecule regulator in an immunization procedure, it would have to undergo extensive preclinical and clinical testing. It may be more efficacious to select a known and well-characterized drug substance that is not otherwise administered to the specific population targeted for immunization. Alternatively, a known drug substance may be selected as a small-molecule regulator that (1) will not need to be administered to subjects within at least the first several weeks after immunization and (2) is indicated only for short-term, sporadic administration, preferably under medical supervision. Thus, a potential low-level risk is further reduced by the avoidance of administration of the drug substance during the period during which immunizing virus is systemically present. Sporadic use of the drug substance under medical supervision will ensure that significant inadvertent replication of reactivated immunizing virus would be rapidly diagnosed and antiviral measures could be taken without delay. In the example systems described herein, the arming small-molecule regulator is a progesterone receptor (PR) antagonist or antiprogestin, e.g., mifepristone or ulipristal. Mifepristone and ulipristal fulfill the latter requirements of not typically needing to be administered shortly after immunization, and of being used only infrequently (and only in a specific segment of the population), and this only under medical supervision. Mifepristone and ulipristal have excellent safety records.

How immunization using a heat- and small-molecule regulator-activated herpesvirus may be practiced is illustrated in the following specific example. A composition comprising an effective amount of a heat- and small-molecule regulator-activated herpesvirus that contains an expressible gene for a pathogen, e.g., influenza, as in the applications disclosed herein, and an effective amount of a small-molecule regulator is administered to a subject intradermally or subcutaneously. Shortly after administration, a heating pad is activated and applied to the inoculation site by either the subject or the physician. Heating at about 43.5-45.5° C. (temperature of the pad surface that is in contact with the skin) will be for a period of about 10-60 min. The latter heat treatment will trigger one cycle of virus replication. If another round of replication is desired, another (or a regenerated) activated pad is applied to the administration site at an appropriate later time. If an immunization procedure involves sequential heat treatments, small-molecule regulator may also need to be administered repeatedly. Alternatively, a sustained release formulation may be utilized that assures the presence of an effective concentration of small-molecule regulator in the administration site region over the period during which viral replication is intended to occur.

More generally, a body region to which a heat- and small-molecule regulator-activated herpesvirus is administered, i.e., the administration site, may be heated by any suitable method. Heat may be delivered or produced in the targeted region by different means including direct contact with a heated surface or a heated liquid, ultrasound, infrared radiation, or microwave or radiofrequency radiation. As proposed in the above specific example, a practical and inexpensive solution may be offered by heating pads (or similar devices of other shapes, e.g., cylinders or cones, for heating mucosal surfaces of the nose, etc.) containing a supercooled liquid that can be triggered by mechanical disturbance to crystallize, releasing heat at the melting point temperature of the chemical used. A useful chemical salt is sodium thiosulfate pentahydrate that has a melting point of about 48° C. U.S. Pat. Nos. 3,951,127, 4,379,448, and 4,460,546. Applicant designed a heating pad using the latter salt (detailed in the example section). When applied to an extremity of a human subject, the activated pad maintained a temperature of 45° C. (+1-0.5° C.) over a period of 15 min. This heat treatment was sufficient to strongly activate in the heat-exposed cells the endogenous HSP70B promoter, which is the same heat shock promoter that is present in applicant's heat- and small-molecule regulator-activated herpesviruses.

In general, an “activating heat dose” (or a “replication-activating heat dose”) is a heat dose that causes a transient activation of HSF1 in cells within the inoculation site region. Activation of this transcription factor is evidenced by a detectably increased level of RNA transcripts of a heat-inducible heat shock gene over the level present in cells not exposed to the heat dose. Alternatively, it may be evidenced as a detectably increased amount of the protein product of such a heat shock gene. More importantly, an activating heat dose may be evidenced by the occurrence of replication of a heat- and small-molecule regulator-activated herpesvirus in the presence of an effective concentration of an appropriate small-molecule regulator.

An activating heat dose can be delivered to the vaccine administration site region at a temperature between about 41° C. and about 47° C. for a period of between about 1 min and about 180 min. It is noted that heat dose is a function of both temperature and time of exposure. Hence, similar heat doses can be achieved by a combination of an exposure temperature at the lower end of the temperature range and an exposure time at the upper end of the time range, or an exposure temperature at the higher end of the temperature range and an exposure time at the lower end of the time range. Preferably, heat exposure will be at a temperature between about 42° C. and about 46° C. for a period of between about 5 min and about 150 min. Most preferably, heat treatment is administered at a temperature between about 43.5° C. and about 45.5° C. for a period of between about 10 min and about 60 min.

An effective (enabling) concentration of a small-molecule regulator in the inoculation site region is a concentration that enables replication (one round) of a heat- and small-molecule regulator-activated herpesvirus in infected cells of that region that have also received an activating heat dose. What an effective concentration is depends on the affinity of the small-molecule regulator for its target transactivator. How such effective concentration is achieved and for how long it is maintained also depends on the pharmacokinetics of the particular small-molecule regulator, which in turn depends on the route of administration of the small-molecule regulator, the metabolism and route of elimination of the small-molecule regulator, the subject being examined, i.e., the type of subject (human or other mammal), its age, condition, weight, etc. It further depends on the type of composition administered, i.e., whether the composition permits an immediate release or a sustained release of the regulator. For a number of well-characterized small-molecule regulator-transactivator systems, effective concentrations in certain experimental subjects have been estimated and are available from the literature. This applies to systems based on progesterone receptors, ecdysone receptors, estrogen receptors, and tetracycline repressor as well as to dimerizer systems, i.e., transactivators activated by rapamycin or analogs (including non-immunosuppressive analogs), or FK506 or analogs. For example, an effective concentration of mifepristone in rats can be reached by i.p. (intraperitoneal) administration of as little as 5 μg mifepristone per kg body weight (5 μg/kg). Amounts would have to be approximately doubled (to about 10 μg/kg), if the small-molecule regulator is administered orally. Wang, Y. et al. (1994) A regulatory system for use in gene transfer. Proc. Natl. Acad. Sci. USA 91: 8180-84. Such amounts of a small-molecule regulator that, upon administration by the chosen route, result in an effective (or enabling) concentration are referred to as effective amounts of the small-molecule regulator in question. How an effective amount of a small-molecule regulator that results in an enabling concentration (at the site of administration of a vaccine of the present disclosure) can be determined is well within the skills of an artisan and is also addressed in the example section.

In the afore-described specific example, a replication-competent controlled virus of the disclosure (a heat- and small-molecule regulator-activated virus) and an appropriate small-molecule regulator were co-administered in a single composition. Replication-competent controlled virus and small-molecule regulator can also be administered in separate compositions. Topical co-administration of immunizing virus and small-molecule regulator appears advantageous for several reasons, including minimization of potential secondary effects of the small-molecule regulator, further reduction of the already remote possibility that virus may replicate systemically during the immunization period, and minimization of the environmental impact of elimination of small-molecule regulator. Notwithstanding these advantages, the small-molecule regulator may be given by a systemic route, e.g., orally, which may be preferred if a formulation of the drug substance of choice is already available that has been tested for a particular route of administration. The relative timing of administration of a heat- and small-molecule regulator-activated virus, administration of an appropriate heat dose and administration of an effective amount of small-molecule regulator is derivative of the operational requirements of the dual-responsive gene switch control. Typically, administration of the immunizing virus will precede heat treatment. This is because heat activation of heat shock transcription factor (HSF1) is transient, and activated factor returns to an inactive state within at most a few hours after activation. The dual-responsive transactivator gene present in the viral genome must be available for HSF1-mediated transcription during the latter short interval of transcription factor activity. For the regulated viral gene(s) to become available for transcription, the immunizing virus will have had to adsorb to a host cell, enter the cell and unravel to present its genome to the cellular transcription machinery. Although not preferred, it is possible to heat-expose the administration site region immediately after (or even shortly before) administration of the immunizing virus. Typically, the administration site region is heat-exposed at a time between about 30 min and about 10 h after virus administration, although heat treatment may be administered even later. Regarding administration of the small-molecule regulator, there typically will be more flexibility because it will be possible to maintain an effective concentration systemically or specifically in the inoculation site region for one to several days. Consequently, small-molecule regulator can be administered prior to, at the time of or subsequent to virus administration, the only requirement being that the regulator be present in an effective (enabling) concentration at the administration site for the time needed for the target transactivator to fulfill its role in enabling viral replication. Typically, this time will correspond to that required for the completion of a round of induced virus replication. Typically, a round of virus replication will be completed within about one day.

As has been alluded to before, a replication-competent controlled herpesvirus, specifically a heat- and small-molecule regulator-activated herpesvirus, may be induced to replicate once or several times. Replication may be re-induced one to several days after the previous round of replication. For any round of replication to occur, the target cells that are infected with the replication-competent controlled virus need to receive an activating heat dose and the tissue of which the latter cells are part (the administration site region) must contain an enabling concentration of small-molecule regulator.

Immunization using a replication-competent controlled virus, specifically a heat- and small-molecule regulator-activated virus, can be by any suitable route, provided that a fraction of administered replication-competent controlled virus infects cells within a defined region that can be subjected to a localized activating treatment, e.g., a focused/localized heat treatment, and replication of the virus in the latter cells triggers the desired immune response without causing disease or undue discomfort. The site to which immunizing virus is administered may be a cutaneous or subcutaneous region located anywhere on the trunk or the extremities of a subject. Preferably, administration of a composition of the invention comprising a replication-competent controlled virus may be to a cutaneous or subcutaneous region located on an upper extremity of a subject. Administration may also be to the lungs or airways, or a mucous membrane in an orifice of a subject. This includes the nasal mucous membrane of a subject.

Dual-responsive gene switches that can be used in a heat- and small-molecule regulator-activated herpesvirus consist of (1) a gene for a small-molecule regulator-activated transactivator, the gene being functionally linked to a promoter or promoter cassette responsive to heat and the transactivator and (2) a promoter responsive to the transactivator for controlling a target gene. Vilaboa, N. et al. (2005) Novel gene switches for targeted and timed expression of proteins of interest. Mol. Ther. 12: 290-8. FIG. 1 shows an example dual-responsive gene switch (the specific gene switch also being referred to as “SafeSwitch”) incorporating antiprogestin-dependent chimeric transactivator GLP65 (or glp65). This transactivator comprises a DNA-binding domain from yeast transcription factor GAL4, a truncated ligand-binding domain from a human progesterone receptor and a transactivation domain from the human RELA protein. Burcin, M. M. et al. (1999) Adenovirus-mediated regulable target gene expression in vivo. Proc. Natl. Acad. Sci. USA 96: 355-60; Ye, X. et al. (2002) Ligand-inducible transgene regulation for gene therapy. Meth. Enzymol. 346: 551-61. In a cell containing gene switch and target gene, the target gene is not expected to be expressed in the absence of small-molecule regulator, e.g., mifepristone, or an activating heat treatment. When the cell is subjected to a heat treatment of an appropriate intensity, (endogenous) heat shock transcription factor 1 (HSF1) is activated, and transcription from the transactivator gene is initiated. A short time later, HSF1 is de-activated, and HSF1-driven expression of transactivator ceases. In the absence of small-molecule regulator, transactivator synthesized remains inactive and is eventually removed by degradation. However, in the presence of small-molecule regulator, transactivator is activated and mediates expression from its own gene as well as from the target gene. As a consequence, a certain transactivator level is maintained and target protein continues to be synthesized (theoretically) for as long as small-molecule regulator remains present. Its withdrawal/removal causes inactivation of transactivator. Target as well as transactivator gene expression will diminish and eventually cease, and the system will reset itself.

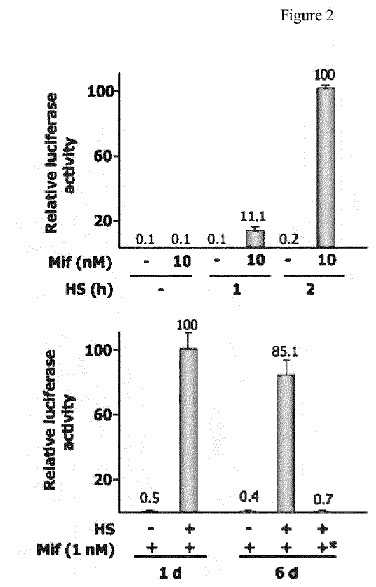

The antiprogestin-armed and heat-activated SafeSwitch was tested extensively in vitro in transient transfection and stable cell line formats and in vivo after electroporation of gene switch and target gene into mouse gastrocnemius muscle. Vilaboa et al. (2005). Results obtained from experiments with a cell line stably containing the gene switch and a luciferase target gene are reproduced in FIG. 2. The data reveal that the system performed as intended. Essentially no target protein expression occurred in the absence of mifepristone, even when cells were subjected to an activating heat treatment. Heat treatment in the presence but not in the absence of mifepristone resulted in activation of target gene expression. It is noted that the heat threshold was relatively elevated: even a 1-h heat treatment at 43° C. only resulted in submaximal activation. Activation of target gene expression was clearly heat dose-dependent. A single heat treatment induced sustained target gene expression for at least 6 days, but only in the continued presence of mifepristone. Removal of mifepristone subsequent to activation resulted in cessation of target gene expression (as evidenced by a disappearance of the labile target gene product).

Analogous dual-responsive gene switches that are activated by heat treatment in the presence of rapamycin or a non-immunosuppressive rapamycin derivative were also developed. Martin-Saavedra, F. M. et al. (2009) Heat-activated, rapamycin-dependent gene switches for tight control of transgene expression. Hum. Gene Ther. 20: 1060-1. Two different versions were prepared that are capable of transactivating a target gene driven by a promoter containing ZFHD1-binding sites (as originally described in Rivera, V. M. et al. (1996) A humanized system for pharmacologic control of gene expression. Nat. Med. 2: 1028-32) or a GAL4 promoter, respectively.

Other examples of small-molecule regulator-activated transactivators than can be incorporated in dual-responsive gene switches or related gene switches include tetracycline/doxycycline-regulated tet-on repressors (Gossen, M. and Bujard, H. (1992) Tight control of gene expression in mammalian cells by tetracycline-responsive promoters. Proc. Natl. Acad. Sci. USA 89, 5547-5551; Gossen, M. et al. (1996) Transcriptional activation by tetracyclines in mammalian cells. Science 268, 1766-1769) and transactivators containing a ligand-binding domain of an insect ecdysone receptor (No, D. et al. (1996) Ecdysone-inducible gene expression in mammalian cells and transgenic mice. Proc. Natl. Acad. Sci. USA 93, 3346-3351). A stringently ligand-dependent transactivator of the latter type is the RheoSwitch transactivator developed by Palli and colleagues. Palli, S. R. et al. (2003) Improved ecdysone receptor-based inducible gene regulation system. Eur. J. Biochem. 270: 1308-15; Kumar, M. B. et al. (2004) Highly flexible ligand binding pocket of ecdysone receptor. A single amino acid change leads to discrimination between two groups of nonsteroidal ecdysone agonists. J. Biol. Chem. 279: 27211-18. The RheoSwitch transactivator can be activated by ecdysteroids such as ponasterone A or muristerone A, or by synthetic diacylhydrazines such as RSL-1 (also known as RH-5849, first synthesized by Rohm and Haas Company). Dhadialla, T. S. et al. (1998) New insecticides with ecdysteroidal and juvenile hormone activity. Annu. Rev. Entomol. 43: 545-69. Other small molecule-regulated transactivators may be used, provided that they can be employed to control the activity of a target gene without also causing widespread deregulation of genes of the host cells and provided further that the associated small-molecule regulators have acceptably low toxicity for the host at their effective concentrations.

Gene switches related to the above-discussed dual-responsive gene switches consist of (1) a gene for a small-molecule regulator-activated transactivator, the gene being functionally linked to a heat-responsive promoter, and (2) a promoter responsive to the transactivator for controlling a gene of interest. Unlike in the above-discussed dual-responsive gene switches, the transactivator gene is not auto-activated. As a consequence, the period of activity of such a gene switch subsequent to a single activating heat treatment is substantially shorter than that of a corresponding dual-responsive gene switch containing an auto-activated transactivator gene.

The example recombinant viruses specifically disclosed herein are derived from HSV-1. Other viruses including other types of herpesviruses can be employed as backbones for construction of a replication-competent controlled virus. These include the alpha herpesviruses HSV-2 and varicella zoster virus (VZV), beta herpesviruses including cytomegalovirus (CMV) and the roseola viruses (HSV6 and HSV7), and gamma herpesviruses such as Epstein-Barr virus (EBV) and Karposi's sarcoma-associated herpesvirus (KSHV). Preferred replication-competent controlled viruses of the present disclosure are derived from HSV-1, HSV2, VZV or CMV viruses.

In a different embodiment, replication-essential genes of a replication-competent controlled virus are not controlled by a dual-responsive gene switch that places them under dual control of heat and a small-molecule regulator, but are individually controlled by a heat shock promoter and a transactivator-responsive promoter, whereby at least one replication-essential gene is controlled by a heat shock promoter and at least one replication-essential gene is controlled by a transactivator-responsive promoter. Transactivator is expressed from a constitutive promoter or from an auto-activated (transactivator-enhanced) promoter. If one replication-essential gene is controlled by a heat shock promoter and another by a promoter responsive to an activated transactivator, replication is also dually controlled by heat and a small-molecule regulator. However, a replication-competent controlled virus of this type is less preferred than a replication-competent controlled virus controlled by a dual-regulated gene switch for several reasons. First, activated HSF1 and, consequently, heat shock promoters tend to be inactivated within a period of a few hours. Hence, if expressed under heat shock promoter control, certain viral genes may not be capable of fulfilling their normal role in the virus replication cycle. Second, also related to the transient nature of the heat shock response, if expression of two differently regulated viral genes (i.e., regulated by heat shock or transactivator, respectively) is required at different times in the virus life cycle, activating heat treatment and small-molecule regulator may need to be administered at different times, adding considerable inconvenience to an immunization procedure. A requirement that only genes that exhibit closely similar expression profiles be selected for regulation would represent a significant constraint on the design of a controlled virus. Finally, in the presence of one of the activating stimuli, i.e., heat or small-molecule regulator, one of the two differently regulated replication-essential genes will become activated, weakening the replication block. In the case where both replication-essential genes are dually regulated, neither of the genes will become active in the presence of small-molecule regulator alone or if the host cell is exposed to heat in the absence of small-molecule regulator. Hence, the stringent inhibition of replication will be maintained even in the presence of one of the activating stimuli. Regarding an appropriate heat dose for activation, nature and properties of transactivators, and properties and effective concentrations of virus and small-molecule regulators, etc., the reader is referred back to earlier sections of this specification.

In yet another less preferred embodiment, one or more replication-essential genes of a replication-competent controlled virus are not dually controlled by heat and a small-molecule regulator, but are singly controlled by a small-molecule regulator. A gene for a small-molecule regulator-activated transactivator is expressed from a constitutive promoter or from an auto-activated promoter. To achieve localization of virus replication, small-molecule regulator is administrated to the site of virus administration, either together with the immunizing virus or separately. A sustained release formulation may be utilized that assures the presence of an effective concentration of small-molecule regulator in the virus administration site for the period during which viral replication is desired. Regarding the nature and properties of transactivators, and properties and effective concentrations of virus and small-molecule regulators, etc., the reader is referred back to earlier sections of this specification.

In the uses and methods of the present disclosure, replication-competent controlled herpesviruses are utilized as vectors to deliver one or more antigens from one or more other infectious agents (other than herpesviruses). Viruses of the herpesviridae family can accommodate sizeable DNA insertions in their genome, which insertions are not expected to reduce significantly replication efficiency. Inserted genes encoding, e.g., influenza virus surface antigens or internal proteins, HIV envelope or internal antigens, etc., may be subjected to heat and/or small molecule regulator control. This would link antigen expression to virus replication and restrict it to the inoculation site region. Alternatively, inserted genes may be placed under the control of other promoters, e.g., constitutive promoters, allowing for expression in non-productively infected cells, which may result in longer periods of antigen expression as well as expression in virus-infected cells outside of the administration site region.

Preferred antigens to be expressed from a replication-competent controlled herpesvirus are proteins or protein fragments from an influenzavirus A or an influenzavirus B. The influenzaviruses contain eight-segmented, negative-sense, single-stranded RNA genomes. Influenzaviruses A genomes encode 11 proteins: polymerase basic subunit 2 (PB2), polymerase basic subunit 1 (PB1), polymerase acidic subunit (PA), hemagglutinin (HA), nucleoprotein (NP), neuraminidase (NA), matrix protein (M1), ion channel protein (M2), nonstructural protein 1 (NS1), nonstructural protein 2 (NS2), and in some strains additional protein PB1-F2. Influenzavirus B has similar proteins, except that the M2 protein is replaced by proteins NB and BM2. Protein fragments can be sizeable portions of a viral protein, e.g., the ectodomain of M2 or a stalk region of an HA. They can also be peptides and small polypeptides that contain sequences of one or more epitopes. Also encompassed are fusions of two or more different viral proteins, two or more viral proteins from different viral strains, or two or more epitopes from one or more viral proteins (different viral proteins or proteins of different strains). Further encompassed are fusions of a viral protein or a fragment thereof and a non-influenza partner protein.

Type A influenza viruses are further divided into subtypes based on the antigenicity of the hemagglutinin (H or HA) and neuraminidase (N or NA) surface glycoproteins. Currently, 18 HA (H1-H18) and 11 NA (N1-N11) subtypes are known, all of which exist in aquatic birds that are their natural reservoirs. Current subtypes of influenza A viruses found in people are influenza A (H1N1) and influenza A (H3N2) viruses. Influenza A viruses can be further broken down into different strains. Influenza B viruses are not divided into subtypes but can be further broken down into lineages and strains. Currently circulating influenza B viruses belong to one of two lineages: B/Yamagata and B/Victoria. Bouvier, N. M., Palese, P. (2008) The biology of influenza viruses. Vaccine 26 (Suppl 4): D49-D53; Du, L., Zhou, Y., Jiang, S. (2010) Research and development of universal influenza vaccines. Microbes & Infection 12: 280-286; www.who.int/immunization/research/meetings_workshops/Universal_lnfluenza_Vaccine RD_Sept2014.pdf “Status of Vaccine Research and Development of Universal Influenza Vaccine. Prepared for WHO PD-VAC”.

Typically, influenza virus strains are characterized by the criteria defined by the WHO in a 1980 memorandum (A revision of the system of nomenclature for influenza viruses. Bulletin of the World Health Organization 58(4): 585-591 (1980)): (1) the antigenic type (e.g., A, B, C), (2) The host of origin (e.g., swine, equine, chicken, etc. For human-origin viruses, no host of origin designation is given), (3) Geographical origin (e.g., Denver, Taiwan, etc.), (4) Strain number (e.g., 15, 7, etc.), (5) Year of isolation (e.g., 57, 2009, etc.), and (6) For influenza A viruses, the hemagglutinin and neuraminidase antigen description in parentheses (e.g., (H1N1), (H5N1). Examples: A/duck/Alberta/35/76 (H1N1) for a virus from duck origin; A/Perth/16/2009 (H3N2) for a virus from human origin.

Influenzavirus A and influenzavirus B strains can be further differentiated into different clades. See, e.g., Tewawong N, et al. (2015) Assessing Antigenic Drift of Seasonal Influenza A(H3N2) and A(H1N1)pdm09 Viruses. PLoS ONE 10(10): e0139958; WHO/OIE/FAO H5N1 Evolution Working Group (2012), Continued evolution of highly pathogenic avian influenza A (H5N1): updated nomenclature. Influenza and Other Respiratory Viruses, 6: 1-5.

A concern has been whether pre-existing immunity to a virus will preclude its use as a vaccine vector. The issue of pre-existing immunity to herpesviruses has been examined in multiple studies. Brockman, M. A. and Knipe, D. M. (2002) Herpes simplex virus vectors elicit durable immune responses in the presence of preexisting host immunity. J. Virol. 76: 3678-87; Chahlavi, A. et al. (1999) Effect of prior exposure to herpes simplex virus 1 on viral vector-mediated tumor therapy in immunocompetent mice. Gene Ther. 6: 1751-58; Delman, K. A. et al. (2000) Effects of preexisting immunity on the response to herpes simplex-based oncolytic therapy. Hum. Gene Ther. 11: 2465-72; Hocknell, P. K. et al. (2002) Expression of human immunodeficiency virus type 1 GP120 from herpes simplex virus type 1-derived amplicons result in potent, specific, and durable cellular and humoral immune responses. J. Virol. 76: 5565-80; Lambright, E. S. et al. (2000) Effect of preexisting anti-herpes immunity on the efficacy of herpes simplex viral therapy in a murine intraperitoneal tumor model. Mol. Ther. 2: 387-93; Herrlinger, U. et al. (1998) Pre-existing herpes simplex virus 1 (HSV-1) immunity decreases but does not abolish, gene transfer to experimental brain tumors by a HSV-1 vector. Gene Ther. 5: 809-19; Lauterbach, H. et al. (2005) Reduced immune responses after vaccination with a recombinant herpes simplex virus type 1 vector in the presence of antiviral immunity. J. Gen. Virol. 86: 2401-10; Watanabe, D. et al. (2007) Properties of a herpes simplex virus multiple immediate-early gene-deleted recombinant as a vaccine vector. Virology 357: 186-98. A majority of these studies reported little effect or only relatively minor effects on immune responses to herpesvirus-delivered heterologous antigens or on anti-tumor efficacy of oncolytic herpesviruses. Brockman, M. A. and Knipe, D. M. (2002); Chahlavi, A. et al. (1999); Delman, K. A. et al. (2000); Hocknell, P. K. et al. (2002); Lambright, E. S. et al. (2000); Watanabe, D. et al. (2007). Two studies were identified that reported substantial reductions of immune responses. Herrlinger, U. et al. (1998); Lauterbach, H. et al. (2005). However, it appears that the results of these studies may not be generalized because inadequate models were employed. One of the studies employed a tumor model that was only barely infectable with the mutant HSV strain used. Herrlinger, U. et al. (1998). The other study employed a chimeric mouse immune model in combination with a severely crippled HSV strain (ICP4⁻, ICP22⁻, ICP27⁻, VHS⁻) as the test vaccine. Lauterbach, H. et al. (2005). All studies agreed that vaccine uses of herpesviruses are possible even in the presence of pre-existing immunity.

Viruses have evolved a multitude of mechanisms for evading immune detection and avoiding destruction. Tortorella, D. et al. (2000) Viral subversion of the immune system. Annu. Rev. Immunol. 18: 861-926. Elimination or weakening of some of these mechanisms could further enhance the immunogenicity of an immunizing virus or viral vector. For example, HSV-1 and HSV-2 express protein ICP47. This protein binds to the cytoplasmic surfaces of both TAP1 and TAP2, the components of the transporter associated with antigen processing TAP. Advani, S. J. and Roizman, B. (2005) The strategy of conquest. The interaction of herpes simplex virus with its host. In: Modulation of Host Gene Expression and Innate Immunity by Viruses (ed. P. Palese), pp. 141-61, Springer Verlag. ICP47 specifically interferes with MHC class I loading by binding to the antigen-binding site of TAP, competitively inhibiting antigenic peptide binding. Virus-infected human cells are expected to be impaired in the presentation of antigenic peptides in the MHC class I context and, consequently, to be resistant to killing by CD8⁺ CTL. Deletion or disablement of the gene that encodes ICP47 ought to significantly increase the immunogenicity of the immunizing virus.

The role of ICP47 has been difficult to study in rodent models, because the protein is a far weaker inhibitor of mouse TAP than of human TAP. Still, one study was able to demonstrate that an HSV-1 ICP47⁻ mutant was less neurovirulent than the corresponding wild type strain and that this reduced neurovirulence was due to a protective CD8 T cell response. Goldsmith, K. et al. (1998) Infected cell protein (ICP)₄₇ enhances herpes simplex virus neurovirulence by blocking the CD8⁺ T cell response. J. Exp. Med. 187: 341-8. Latently infected neurons may exhibit infrequent but detectable expression of viral proteins. Feldman, L. T. et al. (2002) Spontaneous molecular reactivation of herpes simplex virus type 1 latency in mice. Proc. Natl. Acad. Sci. USA 99: 978-83. These proteins may be presented by MHC class I to specific CD8 T cells whose role it may be to prevent virus reactivation. Khanna, K. M. et al. (2004) Immune control of herpes simplex virus during latency. Curr. Opin. Immunol. 16: 463-69. Co-localization of CD8 T cells with infected cells in trigeminal ganglia has been observed. Khanna, K. M. et al. (2003) Herpes simplex virus-specific memory CD8 T cells are selectively activated and retained in latently infected sensory ganglia. Immunity 18: 593-603. That CD8 T cells control virus reactivation from latency and that this control is dependent on MHC class I presentation was demonstrated in a mouse study using HSV-1 recombinants that expressed cytomegalovirus MHC class I inhibitors. Orr, M. T. et al. (2007) CD8 T cell control of HSV reactivation from latency is abrogated by viral inhibition of MHC class I. Cell Host Microbe 2: 172-80. Hence, deletion of ICP47 is expected not only to enhance the immunogenicity of a replication-competent controlled virus but also to reduce the already low probability of its inadvertent reactivation from latency.

The immunogenicity of a herpesvirus vector (i.e., a replication-competent controlled herpesvirus) may also be enhanced by including in the viral genome an expressible gene for a cytokine or other component of the immune system. A vaccination study in mice in which replication-defective herpesvirus recombinants expressing various cytokines were compared demonstrated that virus-expressed IL-4 and IL-2 had adjuvant effects. Osiorio, Y., Ghiasi, H. (2003) Comparison of adjuvant efficacy of herpes simplex virus type 1 recombinant viruses expressing T_{H}1 and T_{H}2 cytokine genes. J. Virol. 77: 5774-83. Further afield, modulation of dendritic cell function by GM-CSF was shown to enhance protective immunity induced by BCG and to overcome non-responsiveness to a hepatitis B vaccine. Nambiar, J. K. et al. (2009) Modulation of pulmonary DC function by vaccine-encoded GM-CSF enhances protective immunity against Mycobacterium tuberculosis infection. Eur. J. Immunol. 40: 153-61; Chou, H. Y. et al. (2010) Hydrogel-delivered GM-CSF overcomes nonresponsiveness to hepatitis B vaccine through recruitment and activation of dendritic cells. J. Immunol. 185: 5468-75.

An “effective amount” of a herpesvirus-vectored vaccine or a replication-competent controlled herpesvirus expressing an antigen of a pathogen (other than a herpesvirus) is an amount of such virus that upon administration to a subject followed by localized activation (typically, at the site of administration) detectably enhances a subject's immune response to the expressed heterologous antigen including resistance to infection by the pathogen, from which the antigen expressed by the virus has been derived, and/or detectable reduction of disease severity, disease duration or mortality subsequent to infection of the vaccinated subject with said pathogen. It is noted that a number of factors will influence what constitutes an effective amount of a herpesvirus-vectored vaccine (i.e., a replication-competent controlled herpesvirus expressing an antigen of a pathogen), including to some extent the site and route of administration of the virus to a subject as well as the activation regimen utilized (i.e., the relative timing of heating and small-molecule regulator administration, the heat dose(s) delivered to the administration site region, the number of replication cycles induced, etc.). Effective amounts of a replication-competent controlled virus will be determined in dose-finding experiments. Generally, an effective amount can be from about 10² to about 10⁸ plaque-forming units (pfu) of virus. More preferably, an effective amount will be from about 10³ to about 10⁷ pfu of virus. Depending on other conditions, an effective amount of a replication-competent controlled virus of the invention may be outside of the above ranges.

A composition or vaccine composition of the present disclosure will comprise effective amounts of one or more herpesvirus-vectored vaccines (i.e., replication-competent controlled herpesviruses expressing an antigen of a pathogen other than a herpesvirus) and, if a small-molecule regulator is also administered as part of the composition, an effective amount of the small-molecule regulator. Although it may be administered in the form of a fine powder under certain circumstances (as disclosed, e.g., in U.S. Pat. Appl. Publ. No 20080035143), the composition typically is an aqueous solution comprising the herpesvirus-vectored vaccine(s) and, as the case may be, a small-molecule regulator. It may be administered to a subject as an aqueous solution or, in the case of administration to a mucosal membrane (nose, lung), as an aerosol thereof. See, e.g., U.S. Pat. No. 5,952,220. The compositions of the present invention will typically include a buffer component. The compositions will have a pH that is compatible with the intended use and is typically between about 6 and about 8. A variety of conventional buffers may be employed such as phosphate, citrate, histidine, Tris, Bis-Tris, bicarbonate and the like and mixtures thereof. The concentration of buffer generally ranges from about 0.01 to about 0.25% w/v (weight/volume).

The compositions may further include, for example, suitable preservatives, virus stabilizers, tonicity agents and/or viscosity-increasing substances. As mentioned before, they may also include an appropriate small-molecule regulator, or a formulation comprising such small-molecule regulator. Preservatives may be present in compositions comprising a herpesvirus-vectored vaccine at concentrations at which they do not or only minimally interfere with the infectivity and replicative efficiency of the virus.

Osmolarity can be adjusted with tonicity agents to a value that is compatible with the intended use of the vaccine compositions. For example, the osmolarity may be adjusted to approximately the osmotic pressure of normal physiological fluids, which is approximately equivalent to about 0.9% w/v of sodium chloride in water. Examples of suitable tonicity adjusting agents include, without limitation, chloride salts of sodium, potassium, calcium and magnesium, dextrose, glycerol, propylene glycol, mannitol, sorbitol and the like, and mixtures thereof. Preferably, the tonicity agent(s) will be employed in an amount to provide a final osmotic value of 150 to 450 mOsm/kg, more preferably between about 220 and about 350 mOsm/kg and most preferably between about 270 and about 310 mOsm/kg.

If indicated, the compositions can further include one or more viscosity-modifying agents such as cellulose polymers, including hydroxypropylmethyl cellulose, hydroxyethyl cellulose, ethylhydroxyethyl cellulose, hydroxypropyl cellulose, methyl cellulose, carboxymethyl cellulose, glycerol, carbomers, polyvinyl alcohol, polyvinyl pyrrolidone, alginates, carrageenans, guar, karaya, agarose, locust bean gum, and tragacanth and xanthan gums. Such viscosity modifying components are typically employed in an amount effective to provide the desired degree of thickening. Viscosity-modifying agents may be present in compositions comprising a replication-competent controlled virus at concentrations at which they do not or only minimally interfere with infectivity and replicative efficiency of the virus.

If the composition also contains a small-molecule regulator, an effective amount of such small-molecule regulator can be included in the composition in the form of a powder, solution, emulsion or particle. As also provided before, an effective amount of a small-molecule regulator to be co-delivered with an effective amount of a replication-competent controlled virus will be an amount that yields an effective concentration of small-molecule regulator in the administration site region, which effective concentration enables at least one round of replication of the replication-competent controlled virus in infected cells of that region. To maintain a small-molecule regulator at an effective concentration for a more extended period, i.e., if replication of the virus (i.e., a heat- and small-molecule regulator-activated virus expressing a heterologous antigen) is reinitiated by a second or further heat treatment of the administration site region, the small-molecule regulator may be included in the form of a sustained release formulation (see also below).

Methods for amplifying viruses are well known in the laboratory art. Industrial scale-up has also been achieved. For herpesviruses, see Hunter, W. D. (1999) Attenuated, replication-competent herpes simplex virus type 1 mutant G207: safety evaluation of intracerebral injection in nonhuman primates. J. Virol. 73: 6319-26; Rampling, R. et al. (2000) Toxicity evaluation of replication-competent herpes simplex virus (ICP34.5 null mutant 1716) in patients with recurrent malignant glioma. Gene Ther. 7: 859-866; Mundle, S. T. et al. (2013) High-purity preparation of HSV-2 vaccine candidate ACAM529 is immunogenic and efficacious in vivo. PLoS ONE 8(2): e57224.

Various methods for purifying viruses have been disclosed. See, e.g., Mundle et al. (2013) and references cited therein; Wolf, M. W. and Reichl, U. (2011) Downstream processing of cell culture-derived virus particles. Expert Rev. Vaccines 10: 1451-75.

While a small-molecule regulator can be co-administered with a herpesvirus-vectored vaccine in a single composition, a composition comprising vaccine and a composition comprising the small-molecule regulator can also be administered separately. The latter composition will comprise an effective amount of a small-molecule regulator formulated together with one or more pharmaceutically acceptable carriers or excipients. A composition comprising a small-molecule regulator may be administered orally, parenterally, by inhalation spray, topically, rectally, nasally, buccally, vaginally or via an implanted reservoir, preferably by oral administration or administration by injection. The compositions may contain any conventional non-toxic, pharmaceutically acceptable carriers, adjuvants or vehicles. In some cases, the pH of the formulation may be adjusted with pharmaceutically acceptable acids, bases or buffers to enhance the stability of the formulated small-molecule regulator or its delivery form. The term parenteral as used in connection with the administration of a small-molecule regulator includes subcutaneous, intracutaneous, intravenous, intramuscular, intraarticular, intraarterial, intrasynovial, intrasternal, intrathecal, intralesional and intracranial injection or infusion techniques.

Liquid dosage forms of a small-molecule regulator for oral administration include pharmaceutically acceptable emulsions, microemulsions, solutions, suspensions, syrups and elixirs. In addition, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include, e.g., wetting agents, emulsifying and suspending agents, sweetening, flavoring, and perfuming agents.

Injectable preparations, for example sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic, parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a small-molecule regulator, it may be desirable to slow the absorption of the compound from, e.g., subcutaneous (or, possibly, intracutaneous) or intramuscular injection. This may be accomplished by the use of a liquid suspension of crystalline or amorphous material with poor water solubility. The rate of absorption of the small-molecule regulator then depends upon its rate of dissolution, which, in turn, may depend upon crystal size and crystalline form. Alternatively, delayed absorption of a parenterally administered small-molecule regulator is accomplished by dissolving or suspending the compound in an oil vehicle. Injectable depot forms are made by forming microcapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the small-molecule regulator with suitable non-irritating excipients or carriers such as cocoa butter, polyethylene glycol or a suppository wax which are solid at ambient temperature but liquid at body temperature and therefore melt in the rectum or vaginal cavity and release the small-molecule regulator.

Solid dosage forms for oral administration of a small-molecule regulator include capsules, tablets, pills, powders, and granules. In such solid dosage forms, the small-molecule regulator is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the small-molecule regulator only, or preferentially, in a certain part of the intestinal tract, optionally, in a delayed manner. Examples of embedding compositions that can be used include polymeric substances and waxes.

Dosage forms for topical or transdermal administration of a small-molecule regulator include ointments, pastes, creams, lotions, gels, powders, solutions, sprays, inhalants or patches. The small-molecule regulator is admixed under sterile conditions with a pharmaceutically acceptable carrier and any preservatives or buffers as may be required.

The ointments, pastes, creams and gels may contain, in addition to a small-molecule regulator, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the small-molecule regulator, excipients such as lactose, talc, silicic acid, aluminum hydroxide, calcium silicates and polyamide powder, or mixtures of these substances. Sprays can additionally contain customary propellants such as chlorofluorohydrocarbons or functional replacements thereof.

Transdermal patches have the added advantage of providing controlled delivery of a compound to the body. Such dosage forms can be made by dissolving or dispensing the compound in the proper medium. Absorption enhancers can also be used to increase the flux of the compound across the skin.

For pulmonary delivery, a composition comprising an effective amount of a small-molecule regulator of the invention is formulated and administered to the subject in solid or liquid particulate form by direct administration, e.g., inhalation into the respiratory system. Solid or liquid particulate forms of the small-molecule regulator prepared for pulmonary deposition include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. Nos. 5,767,068 and 5,508,269, and WO 98/43650). A discussion of pulmonary delivery of antibiotics is also found in U.S. Pat. No. 6,014,969.

What an effective amount of a small-molecule regulator is will depend on the activity of the particular small-molecule regulator employed, the route of administration, time of administration, the distribution, stability and rate of excretion of the particular small-molecule regulator as well as the nature of the specific composition administered. It may also depend on the age, body weight, general health, sex and diet of the subject, other drugs used in combination or contemporaneously with the specific small-molecule regulator employed and like factors well known in the medical arts.

Ultimately, what is an effective amount of a small-molecule regulator has to be determined in dose-finding experiments, in which replication of the herpesvirus-vectored vaccine of interest is assessed experimentally in the administration site region. Once an effective amount has been determined in animal experiments, it may be possible to estimate a human effective amount. “Guidance for Industry. Estimating the maximum safe starting dose for initial clinical trials for therapeutics in adult healthy volunteers”, U.S. FDA, Center for Drug Evaluation and Research, July 2005, Pharmacology and Toxicology. For example, as estimated from rat data, an effective human amount of orally administered mifepristone (for enabling a single cycle of virus replication) will be between about 1 and about 100 μg/kg body weight.

If only a single round of replication of a heat- and small-molecule regulator-controlled herpesvirus vaccine is desired, the small-molecule regulator will be administered to a subject as a single dose. However, the same amount may also be administered to a subject in divided doses. As discussed before, if multiple rounds of virus replication are desired to be induced within a relatively short period, an effective amount of a small-molecule regulator may be administered (once) in a slow/sustained release formulation that is capable of maintaining an effective concentration of small-molecule regulator for the period in question. Alternatively, an effective amount of a small-molecule regulator (that is capable of sustaining a single round of virus replication) can be administered repeatedly, whereby each administration is coordinated with the heat activation of virus replication.

Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as reference to an element or elements is intended to provide support for a similar aspect or embodiment of the invention that “consists of’,” “consists essentially of” or “substantially comprises” that particular element or elements, unless otherwise stated or clearly contradicted by context (e. g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention, thus generally described, may be understood more readily by reference to the following examples, which are provided by way of illustration and are not intended to be limiting of the present invention.

### EXAMPLES

### Example 1: Construction of Replication-Competent Controlled Herpesviruses and Replication-Competent Controlled Herpesviruses Containing an Expressible Gene for an Antigen of Pathogen (Other than a Herpesvirus)

All vectors were constructed using wild type HSV-1 strain 17syn+ as the backbone. This strain is fully virulent, is well characterized, and the complete genomic sequence is available. The generation of the viral recombinants was performed by homologous recombination of engineered plasmids along with purified virion DNA into rabbit skin cells (RS) by the calcium phosphate precipitation method as previously described. Bloom, D. C. 1998. HSV Vectors for Gene Therapy. Methods Mol. Med. 10: 369-386. All plasmids used to engineer the insertions of transactivators, GAL4-responsive promoters and heterologous antigen genes for recombination into the HSV-1 genome were cloned from HSV-1 strain 17syn+. Plasmid IN994 was created as follows: an HSV-1 upstream recombination arm was generated by amplification of HSV-1 DNA (17+) (from base pairs 95,441 to 96,090) with DB112 (5′GAG CTC ATC ACC GCA GGC GAG TCT CTT3′) (SEQ ID NO: 1) and DB113 (5′GAG CTC GGT CTT CGG GAC TAA TGC CTT3′) (SEQ ID NO: 2). The product was digested with SacI and inserted into the SacI restriction site of pBluescript to create pUP. An HSV-1 downstream recombination arm was generated using primers DB115-KpnI (5′GGG GTA CCG GTT TTG TTT TGT GTG AC3′) (SEQ ID NO: 3) and DB120-KpnI (5′GGG GTA CCG GTG TGT GAT GAT TTC GC3′) (SEQ ID NO: 4) to amplify HSV-1 (17+ strain) genomic DNA sequence between base pairs 96,092 and 96,538. The PCR product was digested with KpnI, and cloned into KpnI digested pUP to create pIN994, which recombines with HSV-1 at the intergenic UL43/44 region.

HSV-GS1 contains a transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats. A first recombination plasmid pIN:TA1 was constructed by inserting a DNA segment containing a GLP65 gene under the control of a promoter cassette that combined a human HSP70B promoter and a GAL4-responsive promoter (described in Vilaboa et al. 2005) into the multiple cloning site of plasmid pIN994, between flanking sequences of the HSV-1 UL43 and UL44 genes. The TA cassette was isolated from plasmid Hsp70/GAL4-GLP65 (Vilaboa et al. 2005) and was cloned by 3-piece ligation to minimize the region that was amplified by PCR. For the left insert, Hsp70/GAL4-GLP65 was digested with BamHI and BstX1 and the resulting 2875 bp band was gel purified. This fragment contains the HSP70/GAL4 promoter cassette as well as the GAL4 DNA-binding domain, the progesterone receptor ligand-binding domain and part of the P65 activation domain of transactivator GLP65. The right insert was generated by amplifying a portion of pHsp70/GAL4-GLP65 with the primers TA.2803-2823.fwd (5′TCG ACA ACT CCG ACT TTC AGC3′) (SEQ ID NO: 5) and BGHpA.rev (5′ CTC CTC GCG GCC GCA TCG ATC CAT AGA GCC CAC CGC ATC C3′) (SEQ ID NO: 6). The 763 bp PCR product was digested with BstX1 and NotI, and the resultant 676 bp band was gel-purified. This band contained the 3′end of the p65 activation domain and the BGHpA. For the vector, pIN994 was digested with BamHI and NotI, and the resulting 4099 bp fragment was gel-purified and shrimp alkaline phosphatase (SAP)-treated. The two inserts were then simultaneously ligated into the vector, creating an intact TA cassette. Subsequent to transformation, colony #14 was expanded, and the plasmid was verified by restriction enzyme analysis and then by sequence analysis.

One μg of pIN:TA1 was co-transfected with 2 μg of purified HSV-1 (17syn+) virion DNA into RS cells by calcium phosphate precipitation. The resulting pool of viruses was screened for recombinants by picking plaques, amplifying these plaques on 96 well plates of RS cells, and dot-blot hybridization with a ³²P-labeled DNA probe prepared by labeling a TA fragment by random-hexamer priming. A positive well was re-plaqued and re-probed 5 times and verified to contain the TA by PCR and sequence analysis. This intermediate recombinant was designated HSV-17GS43.

A second recombination plasmid, pBS-KS:GAL4-ICP4, was constructed that contained a GAL4-responsive promoter inserted in place of the native ICP4 promoter by cloning it in between the HSV-1 ICP4 recombination arms in the plasmid pBS-KS:ICP4Δpromoter. This placed the ICP4 transcript under the control of the exogenous GAL4 promoter. This particular promoter cassette includes six copies of the yeast GAL4 UAS (upstream activating sequence), the adenovirus E1B TATA sequence and the synthetic intron IVS8. This cassette was excised from the plasmid pGene/v5-HisA (Invitrogen Corp.) with AatII and HindIII, and the resulting 473 bp fragment was gel-purified. For the vector, pBS-KS:ICP4Δpromoter was digested with AatII and HindIII and the resulting 3962 bp fragment gel-purified and SAP-treated. Ligation of these two fragments placed the GAL4 promoter in front of the ICP4 transcriptional start-site. Subsequent to transformation, colony #5 was expanded, test-digested and verified by sequencing.

One μg of pBS-KS:GAL4-ICP4 was co-transfected with 4 μg of purified HSV-17GS43 virion DNA into cells of the ICP4-complementing cell line E5 (DeLuca, N. A. and Schaffer, P. A. 1987. Activities of herpes simplex virus type 1 (HSV-1) ICP4 genes specifying nonsense peptides. Nucleic Acids Res. 15: 4491-4511) by calcium phosphate precipitation. The resulting pool of viruses was screened for recombinants by picking plaques, amplifying these plaques on 96 well plates of E5 cells, and dot-blot hybridization with a ³²P-labeled DNA probe prepared by labeling the GAL4-responsive promoter fragment by random-hexamer priming. A positive well was re-plaqued and re-probed 7 times and verified to contain the GAL4-responsive promoter in both copies of the short repeat sequences by PCR and sequence analysis. This recombinant was designated HSV-GS1.

To obtain pBS-KS:ΔSacI, the SacI site was deleted from the polylinker of plasmid vector pBluescript-KS+, by digesting the plasmid with SacI. The resulting 2954 bp fragment was gel-purified, treated with T4 DNA polymerase to produce blunt ends, re-circularized and self-ligated. Recombination plasmid BS-KS:ICP4Δpromoter was constructed as follows: to generate a first insert, cosmid COS48 (a gift of L. Feldman) was subjected to PCR with the primers HSV1.131428-131404 (5′ CTC CTC AAG CTT CTC GAG CAC ACG GAG CGC GGC TGC CGA CAC G3′) (SEQ ID NO: 7) and HSV1.130859-130880 (5′ CTC CTC GGT ACC CCA TGG AGG CCA GCA GAG CCA GC3′) (SEQ ID NO: 8). The primers placed HindIII and XhoI sites on the 5′ end of the region and NcoI and KpnI sites on the 3′ end, respectively. The 600 bp primary PCR product was digested with HindIII and KpnI, and the resulting 587 bp fragment was gel-purified. Vector pBS-KS:ΔSacI was digested with HindIII and KpnI, and the resulting 2914 bp fragment was gel-purified and SAP-treated. Ligation placed the first insert into the vector's polylinker, creating pBS-KS:ICP4-3′end. To generate a second insert, cosmid COS48 was subjected to PCR with the primers HSV1.132271-132250 (5′ CTC CTC GCG GCC GCA CTA GTT CCG CGT GTC CCT TTC CGA TGC3′) (SEQ ID NO: 9) and HSV1.131779-131800 (5′ CTC CTC CTC GAG AAG CTT ATG CAT GAG CTC GAC GTC TCG GCG GTA ATG AGA TAC GAG C3′) (SEQ ID NO: 10). These primers placed NotI and SpeI sites on the 5′ end of the region and AatII, SacI, NsiI, HindIII and XhoI sites on the 3′ end, respectively. The 549 bp primary PCR product was digested with NotI and XhoI, and the resulting 530 bp band was gel-purified. This fragment also contained the 45 bp OriS hairpin. Plasmid BS-KS:ICP4-3′ end was digested with NotI and XhoI and the resulting 3446 bp band was gel-purified and SAP-treated. Ligation generated pBS-KS:ICP4Δpromoter. The inserts in pBS-KS:ICP4Δpromoter were verified by sequence analysis.

HSV-GS2 contains transactivator (TA) gene cassette inserted into the intergenic region between UL37 and UL38. In addition, the ICP4 promoter has been replaced with a GAL4-responsive promoter (GAL4-binding site-containing minimal promoter) in both copies of the short repeats. A recombination plasmid, pUL37/38:TA, was constructed by inserting a DNA segment containing a GLP65 gene under the control of a promoter cassette that combined a human HSP70B promoter and a GAL4-responsive promoter into the BspE1/AfIII site of plasmid pBS-KS:UL37/38, between flanking sequences of the HSV-1 UL37 and UL38 genes. (Plasmid pBS-KS:UL37/38 contains the HSV-1 UL37/UL38 intergenic region from nt 83,603-84,417.) The TA cassette was isolated from plasmid Hsp70/GAL4-GLP65 (Vilaboa et al. 2005) and was cloned by 3-piece ligation to minimize the region that was amplified by PCR. For the left insert, pHsp70/GAL4-GLP65 was digested with BamHI (filled in) and BstX1 and the resulting 2875 bp band was gel-purified. This fragment contains the Hsp70/GAL4 promoter cassette as well as the GAL4 DNA binding domain, the progesterone receptor ligand-binding domain and part of the P65 activation domain of transactivator GLP65. The right insert was generated by amplifying a portion of pHsp70/GAL4-GLP65 with the primers TA.2803-2823.fwd and BGHpA.rev. The 763 bp PCR product was digested with BstX1 and NotI (filled in), and the resultant 676 bp band was gel-purified. This band contained the 3′end of the P65 activation domain and the BGHpA. For the vector, pBS-KS:UL37/38 was digested with BspE1 and AfIII, and the resulting 3,772 bp fragment was filled in with T4 DNA polymerase, gel-purified and SAP-treated. The two inserts were then simultaneously ligated into the vector, creating an intact TA cassette. Following transformation, colonies were screened by restriction digestion. Colony #29 was expanded, and the plasmid verified by restriction enzyme analysis and then by sequence analysis.

One μg of pUL37/38:TA was co-transfected with 2 μg of purified HSV-1 (17+) virion DNA into RS cells by calcium phosphate precipitation. The resulting pool of viruses was screened for recombinants by picking plaques, amplifying these plaques on 96 well plates of RS cells, and dot-blot hybridization with a ³²P-labeled DNA probe prepared by labeling a TA fragment by random-hexamer priming. A positive well was re-plaqued and re-probed 6 times and verified to contain the TA by PCR and sequence analysis. This intermediate recombinant was designated HSV-17GS38.

One μg of pBS-KS:GAL4-ICP4 was co-transfected with 5 μg of purified HSV-17GS38 virion DNA into E5 cells by calcium phosphate precipitation. The resulting pool of viruses were screened for recombinants by picking plaques, amplifying these plaques on 96 well plates of E5 cells, and dot-blot hybridization with a ³²P-labeled DNA probe prepared by labeling the GAL4-responsive promoter fragment by random-hexamer priming. A positive well was re-plaqued and re-probed 7 times and verified to contain the GAL4-responsive promoter in both copies of the short repeat sequences by PCR and sequence analysis. This recombinant was designated HSV-GS2.

HSV-GS3 contains a transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats. Furthermore, the ICP8 promoter was replaced with a GAL4-responsive promoter. The construction of this recombinant virus involved placing a second HSV-1 replication-essential gene (ICP8) under control of a GAL4-responsive promoter. HSV-GS1 was used as the “backbone” for the construction of this recombinant. ICP8 recombination plasmid pBS-KS:GAL4-ICP8 was constructed. This plasmid contained a GAL4-responsive promoter inserted in place of the native ICP8 promoter by cloning it in between the HSV-1 ICP8 recombination arms in the plasmid pBS-KS:ICP8Δpromoter. This placed the ICP8 transcript under the control of the exogenous GAL4-responsive promoter. This particular promoter cassette consisted of six copies of the yeast GAL4 UAS (upstream activating sequence), the adenovirus E1b TATA sequence and the synthetic intron Ivs8. This cassette was excised from the plasmid pGene/v5-HisA (Invitrogen Corp.) with AatII and HindIII, and the resulting 473 bp fragment gel-purified. For the vector, pBS-KS:ICP8Δpromoter was digested with AatII and HindIII, and the resulting 4588 bp fragment gel-purified and SAP-treated. Ligation of the latter two DNA fragments placed the GAL4-responsive promoter cassette in front of the ICP8 transcriptional start-site. Subsequent to transformation, colony #10 was expanded, test-digested and verified by sequencing.

One μg of pBS-KS:GAL4-ICP8 was co-transfected with 10 μg of purified HSV-GS1 virion DNA into E5 cells by calcium phosphate precipitation. Subsequent to the addition of mifepristone to the medium, the transfected cells were exposed to 43.5° C. for 30 minutes and then incubated at 37° C. Subsequently on days 2 and 3, the cells were again incubated at 43.5° C. for 30 minutes and then returned to 37° C. Plaques were picked and amplified on 96 well plates of E5 cells in media supplemented with mifepristone. The plates were incubated at 43.5° C. for 30 minutes 1 hour after infection and then incubated at 37° C. Subsequently on days 2 and 3, the plates were also shifted to 43.5° C. for 30 minutes and then returned to 37° C. After the wells showed 90-100% CPE, the plates were dot-blotted and the dot-blot membrane hybridized with a ³²P-labeled DNA probe prepared by labeling the HSV-1 ICP8 promoter fragment that was deleted. A faintly positive well was re-plaqued and re-probed 8 times and verified to have lost the ICP8 promoter and to contain the GAL4-responsive promoter in its place by PCR and sequence analysis. This recombinant was designated HSV-GS3.

Recombination plasmid pBS-KS:ICP8Δpromoter was constructed using essentially the same strategy as that described above for the creation of pBS-KS:ICP4Δpromoter: a first insert was PCR-amplified from HSV-1 17syn+ virion DNA using the primers HSV1.61841-61865 (5′ CTC CTC AGA ACC CAG GAC CAG GGC CAC GTT GG3′) (SEQ ID NO: 11) and HSV1.62053-62027 (5′ CTC CTC ATG GAG ACA AAG CCC AAG ACG GCA ACC3′) (SEQ ID NO: 12) and subcloned to yield intermediate vector pBS-KS:ICP8-3′ end. A second insert was similarly obtained using primers HSV1.62173-62203 (5′ CTC CTC GGA GAC CGG GGT TGG GGA ATG AAT CCC TCC3′) (SEQ ID NO: 13) and HSV1.62395-62366 (5′ CTC CTC GCG GGG CGT GGG AGG GGC TGG GGC GGA CC3′) (SEQ ID NO: 14) and was subcloned into pBS-KS:ICP8-3′ end to yield pBS-KS:ICP8Δpromoter.

HSV-GS4: contains a transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats, and the ICP8 promoter has been replaced with a GAL4-responsive promoter. Furthermore, the US12 gene has been mutated to render its protein product (ICP47) nonfunctional. ICP47 amino acid residue K31 was changed to G31, and R32 to G32. Neumann, L. et al. (1997) J. Mol. Biol. 272: 484-92; Galocha, B. et al. (1997) J. Exp. Med. 185: 1565-72. A 500 bp ICP47 coding sequence-containing fragment was PCR-amplified from virion DNA of strain 17syn+. The fragment was PCR-amplified as two pieces (a “left-hand” and a “right-hand” piece), using two primer pairs. The mutations were introduced through the 5′ PCR primer for the right-hand fragment. The resulting amplified left-hand and mutated right-hand fragments were subcloned into vector pBS, and the sequence in subclones was confirmed by sequence analysis. A subclone containing the 500 bp fragment with the desired mutations in ICP47 codons 31 and 32 was termed pBS:mut-ICP47.

One μg of pBS:mut-ICP47 was co-transfected with 10 μg of purified HSV-GS3 virion DNA into E5 cells by calcium phosphate precipitation. Subsequent to the addition of mifepristone to the medium, the transfected cells were exposed to 43.5° C. for 30 minutes and then incubated at 37° C. Subsequently on days 2 and 3, the cells were again incubated at 43.5° C. for 30 minutes and then returned to 37° C. Plaques were picked and amplified on 96 well plates of E5 cells in media supplemented with mifepristone. The plates were incubated at 43.5° C. for 30 minutes 1 hour after infection and then incubated at 37° C. Subsequently on days 2 and 3, the plates were also shifted to 43.5° C. for 30 minutes and then returned to 37° C. After the wells showed 90-100% CPE, the plates were dot-blotted and the dot-blot membrane hybridized with a ³²P-labeled oligonucleotide probe to the mutated ICP47 region. A positive well was re-plaqued and re-probed several times and verified by sequence analysis to contain the expected mutated ICP47 gene sequence. This recombinant was designated HSV-GS4.

HSV-GS5 contains an expressible (auto-activated) transactivator (TA) gene inserted into the intergenic region between UL43 and UL44. In addition, the ICP4 promoter is replaced with a GAL4-responsive promoter (GAL4-binding site-containing minimal promoter) in both copies of the short repeats. A recombination plasmid pIN:TA2 is constructed by inserting a DNA segment containing an auto-activated glp65 gene into the multiple cloning site of plasmid pIN994, between flanking sequences of the HSV-1 UL43 and UL44 genes. The expressible TA gene is isolated from pHsp70/GAL4-GLP65 (Vilaboa et al. 2005) and pSwitch (Invitrogen life technologies), respectfully, and is cloned by 3-piece ligation to minimize the region that is amplified by PCR. For the left insert, pSwitch is digested with SspI and BstX1, and a resulting 2425 bp band is gel purified. This fragment contains the auto-activated promoter as well as the GAL 4 DNA-binding domain, the progesterone receptor ligand-binding domain and part of the P65 activation domain of transactivator GLP65. The right insert is generated by amplifying a portion of pHsp70/GAL4-GLP65 with the primers TA.2803-2823.fwd and BGHpA.rev. The 763 bp PCR product is digested with BstX1 and NotI, and the resultant 676 bp band is gel-purified. This band contains the 3′end of the P65 activation domain and the BGHpA. For the vector, pIN994 is first digested with BamHI, ends are filled in with Klenow DNA polymerase, and the DNA is further digested with NotI. The resulting 4099 bp fragment is gel-purified. The two inserts are then simultaneously ligated into the vector, creating an intact expressible TA gene. Subsequent to transformation, several colonies are expanded and plasmid DNAs subjected to restriction and then sequence analysis to identify pIN:TA2.

One μg of pIN:TA2 is co-transfected with 2 μg of purified HSV-1 virion DNA into RS cells by calcium phosphate precipitation. The resulting pool of viruses is screened for recombinants by picking plaques, amplifying these plaques on 96 well plates of RS cells, and dot-blot hybridization with a ³²P-labeled DNA probe prepared by labeling a TA fragment by random-hexamer priming. A positive well is re-plaqued and re-probed several times and verified to contain the TA by PCR and sequence analysis. This intermediate recombinant is designated HSV-17GS43A.

One μg of pBS-KS:GAL4-ICP4 is co-transfected with 4 μg of purified HSV-17GS43A virion DNA into cells of the ICP4-complementing cell line E5 by calcium phosphate precipitation. The resulting pool of viruses is screened for recombinants by picking plaques, amplifying these plaques on 96 well plates of E5 cells, and dot-blot hybridization with a ³²P-labeled DNA probe prepared by labeling the GAL4-responsive promoter fragment by random-hexamer priming. A positive well is re-plaqued and re-probed several times and verified to contain the GAL4-responsive promoter in both copies of the short repeat sequences by PCR and sequence analysis. This recombinant is designated HSV-GS5.

HSV-GS6 contains an auto-activated transactivator (TA) gene inserted into the intergenic region between UL43 and UL44. In addition, the ICP4 promoter is replaced with a GAL4-responsive promoter (GAL4-binding site-containing minimal promoter) in both copies of the short repeats. Furthermore, the ICP8 promoter is replaced with a human HSP70B promoter. The construction of this recombinant virus involves placing a second HSV-1 replication-essential gene (ICP8) under control of an Hsp70B promoter. HSV-GS5 is used as the “backbone” for the construction of this recombinant. ICP8 recombination plasmid pBS-KS:Hsp70B-ICP8 is constructed that contains an HSP70B promoter inserted in place of the native ICP8 promoter by cloning it in between the HSV-1 ICP8 recombination arms in the plasmid pBS-KS:ICP8Δpromoter. To isolate a human HSP70B promoter fragment, construct p17 is digested with BamHI, ends are filled in by Klenow DNA polymerase, and the DNA is further digested with HindIII. A 450 bp promoter fragment is gel-purified (Voellmy, R. et al. (1985) Proc. Natl. Acad. Sci. USA 82: 4949-53). For the vector, pBS-KS:ICP8Δpromoter is digested with ZraI and HindIII. The resulting 4588 bp fragment is gel-purified. Ligation of the latter two DNA fragments places the HSP70B promoter in front of the ICP8 transcriptional start-site. Subsequent to transformation, several colonies are expanded and plasmid DNAs subjected to restriction and then sequence analysis to identify pBS-KS:HSP70B-ICP8.

One μg of pBS-KS:HSP70B-ICP8 is co-transfected with 10 μg of purified HSV-GS5 virion DNA into E5 cells by calcium phosphate precipitation. The transfected cells are exposed to 43.5° C. for 30 minutes and then incubated at 37° C. Subsequently on days 2 and 3, the cells are again incubated at 43.5° C. for 30 minutes and then returned to 37° C. Plaques are picked and amplified on 96 well plates of E5 cells. The plates are incubated at 43.5° C. for 30 minutes a few hours after infection and then incubated at 37° C. Subsequently on days 2 and 3, the plates are also shifted to 43.5° C. for 30 minutes and then returned to 37° C. After the wells show 90-100% CPE, the plates are dot-blotted and the dot-blot membrane hybridized with a ³²P-labeled DNA probe prepared by labeling the HSP70B promoter fragment by random-hexamer priming. A positive well is re-plaqued and re-probed several times and verified to have lost the ICP8 promoter and to contain the HSP70B promoter in its place by PCR and sequence analysis. This recombinant is designated HSV-GS6.

HSV-GS11 was derived from the vector HSV-GS3 and contains an insertion between the UL37 and UL38 genes of a gene cassette expressing the A/Equine/Prague/1/56 (H7N7) HA gene driven by the CMV IE promoter. The recombination plasmid was constructed by the following sequential steps. First, the 814 bp fragment containing the region spanning the HSV-1 UL37/UL38 intergenic region from nt 83,603-84,417 from the plasmid NK470 was subcloned into pBS that had had the MCS removed (digestion with KpnI/SacI) to yield pBS:UL37/38. A cassette containing a synthetic CMV IE promoter flanked by the pBS-SK+ MCS was ligated into pBS:UL37/38 that was digested with BspE1/AfIII, which cuts between the UL37 and UL38 genes to yield the plasmid pIN:UL37/38. The EIV Prague/56 HA gene was PCR cloned from cDNA prepared from EIV Prague/56. Briefly, RNA was prepared by Trizol extraction of a stock of EIV Prague 56 and reverse transcribed using Omni-Script Reverse Transcriptase (Qiagen) according to the manufacturer's instructions. The cDNAs were cloned into pBS, and the clone containing the HA gene (pBS-EIVPrague56/HA) was confirmed by sequence analysis. The Prague/56 HA gene was excised from this plasmid and inserted behind the CMV promoter in the plasmid pIN:UL37/38 to yield plasmid pIN:37/38-Prague56/HA. To produce recombinant HSV-GS11, RS cells were co-transfected with plasmid pIN:37/38-Prague56/HA and purified HSV-GS3 virion DNA. Subsequent to the addition of mifepristone to the medium, the co-transfected cells were exposed to 43.5° C. for 30 min and then incubated at 37° C. Subsequently, on days 2 and 3, the cells were again incubated at 43.5° C. for 30 min and then returned to 37° C. Picking and amplification of plaques, screening and plaque purification was performed essentially as described for HSV-GS3. The resulting plaque-purified HSV-GS11 was verified by Southern blot as well as by PCR and DNA sequence analysis of the recombination junctions.

HSV-GS10 was derived from the vector HSV-GS3 and contains an insertion between the UL37 and UL38 genes of a gene cassette expressing the A/Equine/Prague/1/56 (H7N7) nucleoprotein gene driven by the CMV IE promoter. HSV-GS12 was derived from the vector HSV-GS3 and contains an insertion between the UL37 and UL38 genes of a gene cassette expressing the A/Equine/Kentucky/1/94 (H3N8) nucleoprotein gene driven by the CMV IE promoter. HSV-GS13 was derived from the vector HSV-GS3 and contains an insertion between the UL37 and UL38 genes of a gene cassette expressing the A/Equine/Kentucky/1/94 (H3N8) HA gene driven by the CMV IE promoter. HSV-GS10, HSV-GS12 and HSV-GS13 were constructed using methods analogous to those described above for HSV-GS11.

Generally known molecular biology and biochemistry methods were used. Molecular biology methods are described, e.g., in “Current protocols in molecular biology”, Ausubel, F. M. et al., eds., John Wiley and Sons, Inc. ISBN: 978-O-471-50338-5.

### Example 2: Regulated Replication of the Replication-Competent Controlled Viruses

### (a) Growth and Replication Properties of HSV-GS1

### Plaque Analysis on Permissive (E5) and Non-Permissive (RS) Cells

Serial dilutions of the purified HSV-GS1 virus were plated onto confluent monolayers of either rabbit skin (RS) or ICP4-complementing Vero-based helper cell line (E5) cells in 60 mm dishes. The virus was allowed to adsorb for 1 hour at 37° C., and then the inoculum was removed, and the cells were overlayed with complete medium (Modified Eagles Medium supplemented with 5% calf serum or 10% fetal bovine serum for RS or E5 cells, respectively). The dishes were then incubated 72 hours and stained with crystal violet to visualize any viral plaques. At dilutions resulting in 10-100 plaques on E5 cells, no plaques were observed on the non-complementing RS cells.

### Growth Analysis of HSV-GS1

Confluent monolayers of either RS or E5 cells in 60 mm dishes were infected as described above with HSV-GS1 at a multiplicity-of-infection (m.o.i.) of 5, incubated for 48 hours, harvested by scraping cells into the medium, frozen at −80° C., subjected to 2 rounds of freezing-thawing and titrated for infectious virus. Results are shown in Table 1.

TABLE 1
Titration of viruses in RS and E5 cells

| Virus | RS cells | E5 cells |
|---|---|---|
| 17syn+ | 8.3 × 10⁸ pfu/ml | 1.2 × 10⁷ pfu/ml |
| HSV-GS1 | ND* | 4.0 × 10⁷ pfu/ml |

*ND = None detected. (Detection limit in this experiment was about 100 pfu.)

### Analysis of the Effects of Heat Exposure and Mifepristone on the Replication of HSV-GS1 in Vero Cells

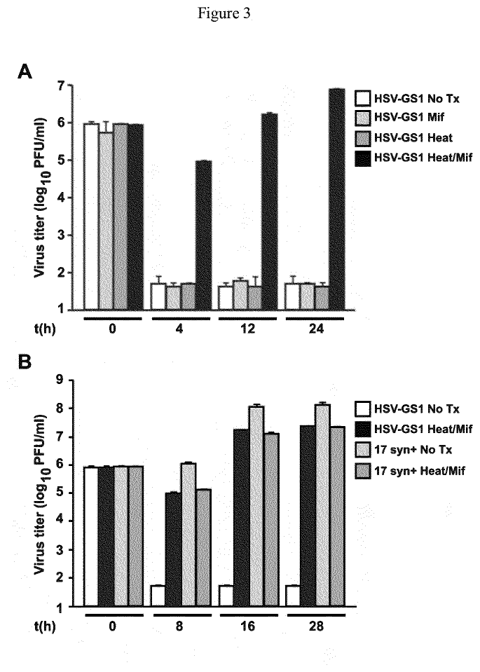

The purpose of this experiment was to compare the replication cycle of HSV-GS1 with the wild type vector HSV-1 strain 17syn+. Confluent monolayers of Vero cells were infected with either HSV-1 strain 17syn+ or the recombinant HSV-GS1 at an m.o.i. of 3. The virus was allowed to adsorb for 1 hour at 37° C., and then the inoculum was removed, and the cells were overlayed with complete medium (Modified Eagles Medium supplemented with 10% fetal bovine serum). Heat treatment was performed 4 hours after adsorption by floating the sealed dishes in a 43.5° C. water bath for 30 minutes. Mifepristone treatment (10 nM) was initiated at the time of the initial infection. The dishes were then incubated for 72 hours at 37° C. At 0, 8, 16 and 28 hours post-infection, two dishes were removed, the cells scraped into the media to harvest, and subjected to 2 freeze-thaw cycles. The infectious virus was then determined by titrating the lysate of each dish in triplicate on 24-well plates of confluent E5 cells. Plaques were visualized after 2 days by staining with crystal violet. Results demonstrate that, under the chosen experimental conditions, HSV-GS1 replicates as efficiently as wild type virus 17syn+(FIG. 3B). No replication of HSV-GS1 appears to occur in the absence of an activation treatment (heat and mifepristone). It is also noted that the activating treatment, i.e., heat exposure and incubation in the presence of mifepristone, only slightly affected wild type virus replication.

Analysis of the Dependence of HSV-GS1 Replication on Both Heat Exposure of the Host Cell and the Presence of Small-Molecule Regulator

The purpose of this experiment was to determine whether the activation of the HSV-GS1 recombinant by heat and mifepristone was due to a requirement of both, and that mifepristone alone or heat alone was not sufficient to induce replication. The experiment was performed as described in the preceding section with the exception that heat treatment was administered immediately after adsorption. The data show that replication of HSV-GS1 did not occur unless the host cells were exposed to heat in the presence of mifepristone (FIG. 3A).

### (b) Growth and Replication Properties of HSV-GS3

### Replication Efficiency of HSV-GS3

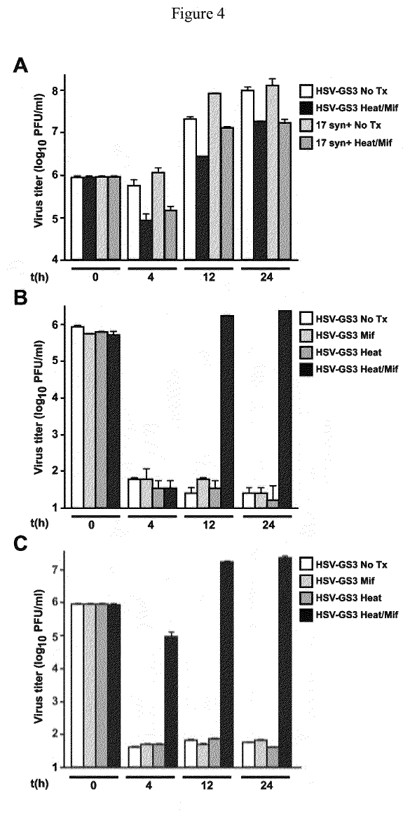

The purpose of this experiment was to compare the replication cycle of the HSV-GS3 recombinant with the wild type vector HSV-1 17syn+. Confluent monolayers of E5 cells (but see below) were infected with either HSV-1 strain 17syn+ or the recombinant HSV-GS3 at an m.o.i. of 3. Virus was allowed to adsorb for 1 hour at 37° C., and then the inoculum was removed and the cells overlayed with complete medium (Modified Eagles Medium supplemented with 10% fetal bovine serum). Heat treatment was performed after adsorption by floating the sealed dishes in a 43.5 C water bath for 30 minutes. Mifepristone treatment (10 nM) was initiated at the time of the initial infection. For the HSV-GS3 No Tx (no treatment) set, the E5 cells were transfected with a plasmid containing an expressible ICP8 gene 12 hours prior to infection. The dishes were then incubated further at 37° C. At 0, 4, 12 and 24 hours post-infection, two dishes were removed, the cells scraped into the media to harvest, and subjected to 2 freeze-thaw cycles. The infectious virus was then determined by titrating the lysate of each dish in triplicate on 24-well plates of confluent E5 cells previously transfected with ICP8 expression plasmid. Plaques were visualized after 2 days by staining with crystal violet. Results indicate that HSV-GS3 replicated nearly as efficiently as wild-type virus HSV-1 17syn+ under the chosen experimental conditions (FIG. 4A).

Analysis of the Dependence of HSV-GS3 Replication on Both Heat Exposure of the Host Cell and the Presence of Small-Molecule Regulator

Single-step growth experiments were carried out to determine whether the activation of HSV-GS3 by heat and mifepristone was due to a requirement of both, and that mifepristone alone or heat alone was not sufficient to induce replication. The experiment shown in FIG. 4B was performed as described in the preceding section with the exception that Vero cells were used. Titrations were in E5 cells previously transfected with ICP8 expression plasmid. A similar experiment was carried out with human squamous cell tumor line SCC-15 (FIG. 4C). The results of the latter experiments demonstrate that replication of HSV-GS3 is tightly regulated. It is only triggered by heat exposure of infected cells in the presence of mifepristone.

In other experiments, measurement of virus replication by titration of infectious virus was substituted by methods of quantification of viral DNA or expression of viral genes. In these experiments mifepristone and/or ulipristal (small-molecule regulators) were tested. One such experiment had the design summarized in Table 2.

TABLE 2
Treatment groups

| | Ulipristal | | | | Mifepristone | No drug |
|---|---|---|---|---|---|---|
| | 0.1 nM | 0.3 nM | 1 nM | 10 nM | 10 nM | — |
| Heat | X | X | X | X | X | X |
| treatment | | | | | | |
| No heat | | | | X | X | X |
| treatment | | | | | | |

Thirty-five mm dishes of confluent Vero cells were infected at an m.o.i. of 3 with the HSV-GS3 vector. Each treatment group consisted of 3 replicate dishes (for each time point). Virus was adsorbed for 1 hour at 37° C., the inoculum removed, and the cells overlayed with complete medium (Modified Eagles Medium supplemented with 10% fetal bovine serum). Drug (i.e., mifepristone or ulipristal) treatment was initiated at the time of the initial infection. Heat treatment was performed after adsorption by floating the sealed dishes in a 43.5° C. water bath for 30 minutes on a submerged platform (initiated 4 hours post infection). Dishes were incubated further at 37° C. At 1, 4, 12 and 24 h post heat treatment, three dishes were removed, the media removed, and the DNA and RNA extracted using TRIzol. Extracted DNA was subjected to Taqman Realtime PCR for quantitative analysis for HSV-1 DNA (using HSV DNA polymerase primers/probe). Extracted RNA was analyzed by Taqman RT-PCR for the presence of ICP4 and glycoprotein C (gC) transcripts. DNA and RNA quantities were normalized relative to the cellular gene APRT and presented as relative quantities. Primers used are shown in Table 3 below.

TABLE 3
Primers and probes used for qPCR

| | | |
|---|---|---|
| HSV DNA | F | 5′ AGAGGGACATCCAGGACTTTGT |
| Pol | | (SEQ ID NO: 15) |
| | R | 5′ CAGGCGCTTGTTGGTGTAC |
| | | (SEQ ID NO: 16) |
| | P | 5′ ACCGCCGAACTGAGCA |
| | | (SEQ ID NO: 17) |
| ICP4 | F | 5′ CACGGGCCGCTTCAC |
| | | (SEQ ID NO: 18) |
| | R | 5′ GCGATAGCGCGCGTAGA |
| | | (SEQ ID NO: 19) |
| | P | 5′ CCGACGCGACCTCC |
| | | (SEQ ID NO: 20) |
| gC | F | 5′ CCTCCACGCCCAAAAGC |
| | | (SEQ ID NO: 21) |
| | R | 5′ GGTGGTGTTGTTCTTGGGTTTG |
| | | (SEQ ID NO: 22) |
| | P | 5′ CCCCACGTCCACCCC |
| | | (SEQ ID NO: 23) |
| Mouse APRT | F | CTCAAGAAATCTAACCCCTGACTCA |
| | | (SEQ ID NO: 24) |
| | R | GCGGGACAGGCTGAGA |
| | | (SEQ ID NO: 25) |
| | P | CCCCACACACACCTC |
| | | (SEQ ID NO: 26) |
| EIV | F | GGAACATTAGAATTCACAGCAGAGG |
| Prague/56 | | (SEQ ID NO: 27) |
| HA | R | CCTGTTCTCAATTTAACATATCCCC |
| | | (SEQ ID NO: 28) |
| | P | GGGGATATGTTAAAT |
| | | (SEQ ID NO: 29) |

F: forward; R: reverse; P: probe.

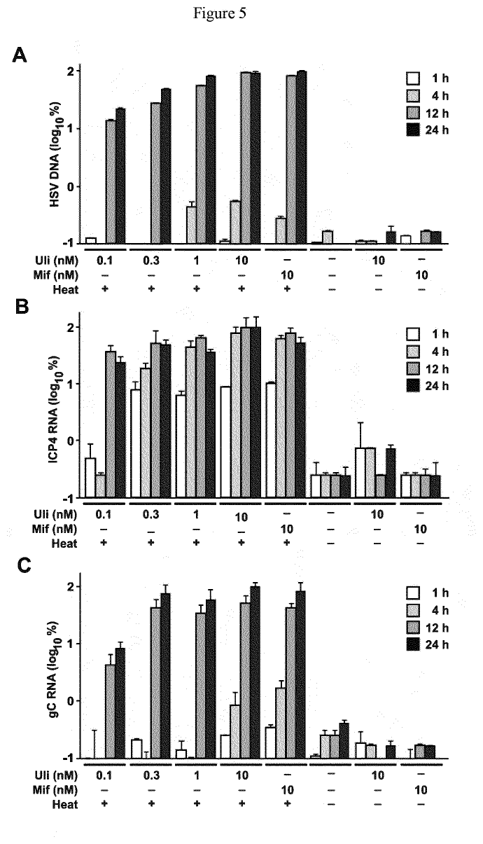

Results obtained are shown in FIG. 5. FIG. 5A shows that viral DNA replication depended both on heat treatment and small-molecule regulator. For ulipristal it was demonstrated that (the rate of) DNA replication was dependent on regulator dose. Compatible data were obtained for the expression of the ICP4 genes (regulated genes) and the late gC gene (not subject to deliberate regulation) (FIGS. 5B & C).

### Example 3: Apparent Inability of a Replication-Competent Controlled Virus to Replicate In Vivo in the Absence of Deliberate Activation

The goal of this experiment was to demonstrate that, in the absence of heat and small-molecule regulator, the GS vectors are as tightly “off” in mice as they appear to be in cell culture. Four to six weeks old out-bred ND4 Swiss Webster female mice (Harlan Sprague-Dawley, Inc.) were infected with similar amounts of HSV-GS1 or 17syn+ wild type virus on the lightly abraded plantar surface of both rear feet following saline pre-treatment. At 4, 8, and 21 days post infection, 4 mice per time point were euthanized and the feet and dorsal root ganglia (DRG) were harvested, homogenized in TRIzol, and DNA and RNA extracted. DNA was subjected to Taqman Realtime PCR for quantitative analysis for HSV-1 DNA; RNA was analyzed following RT by Taqman Realtime PCR for the presence of ICP4 and glycoprotein C (gC) transcripts as previously described. Kubat, N.J. et al. 2004. The herpes simplex virus type 1 latency-associated transcript (LAT) enhancer/rcr is hyperacetylated during latency independently of LAT transcription. J. Virol. 78: 12508-18. The real-time primer/probe sequences are disclosed in Table 3.

Results showed a complete absence of HSV-GS1 gene expression in feet as well as in DRG (Table 4). Consistent with this result implying that HSV-GS1 was incapable of replication was the finding that DNA amounts of HSV-GS1 were orders of magnitude lower than those of wild-type virus HSV 17syn+. Replication efficiency difference at 8 days was 151 fold in feet and 200 fold in DRG, respectively.

TABLE 4
HSV-GS1 and wild type HSV-1: replication and viral gene expression

| Feet: | | | | | | |
|---|---|---|---|---|---|---|
| Time post infection | HSV 17syn+ | | | HSV-GS1 | | |
| | DNA | RNA | | DNA | RNA | |
| | | ICP4 | gC | | ICP4 | gC |
| 4 | 49,500 | 1,290 | 5,742 | 1,222 | ND | ND |
| 8 | 35,773 | 976 | 5,332 | 237 | ND | ND |
| 21 | 49 | ND* | ND | ND | ND | ND |
| DRG: | | | | | | |
| Time post infection | HSV 17syn+ | | | HSV-GS1 | | |
| | DNA | RNA | | DNA | RNA | |
| | | ICP4 | gC | | ICP4 | gC |
| 4 | 12,674 | 576 | 3,563 | 59 | ND | ND |
| 8 | 14,986 | 877 | 9,230 | 75 | ND | ND |
| 21 | 5,754 | ND* | ND | 45 | ND | ND |

*ND = below the limit of detection.

### Example 4: Activation In Vivo of a Replication-Competent Controlled Virus of the Invention

In these experiments, virus replication in a mouse model was estimated by the biochemical methods of quantification of viral DNA and expression of viral genes. DNA amounts and viral transcript amounts were measured at both the foot (site of virus inoculation) and in the dorsal root ganglia (DRG) (site of HSV acute replication and latency). One such experiment had the design summarized in Table 5. This experiment was also aimed at determining the lowest effective in vivo dose of ulipristal.

TABLE 5
Treatment groups

| | Ulipristal | | | No drug |
|---|---|---|---|---|
| | 1 μg/kg | 5 μg/kg | 50 μg/kg | — |
| Heat | X | X | X | X |
| treatment | | | | |
| No heat | | | X | |
| treatment | | | | |

Outbred Swiss-webster female mice (4-6 weeks old) were inoculated with 1×10⁵ pfu of HSV-GS3 vector following saline-pretreatment and light abrasion of both rear footpads. Each treatment group consisted of 5 mice. Drug treatment (ulipristal) was administered i.p. (intraperitoneally) at the time of infection. Heat treatment was performed at 45° C. for 10 min (by immersion of hind feet in a waterbath) 3 h after virus administration. Mice were allowed to recover at 37° C. for 15 min. Mice were sacrificed 24 hours post heat induction, and the feet and DRG were dissected and snap-frozen in RNAlater (Sigma-Aldrich). DNA and RNA were extracted by grinding the tissues in TRIzol (Life Technologies), and back-extracting the DNA from the interface. DNA was subjected to Taqman Realtime PCR for quantitative analysis for HSV-1 DNA. RNA was analyzed following reverse transcription (RT) by Taqman PCR for the presence of ICP4 and glycoprotein C (gC) transcripts. DNA and RNA quantities were normalized relative to the cellular gene APRT.

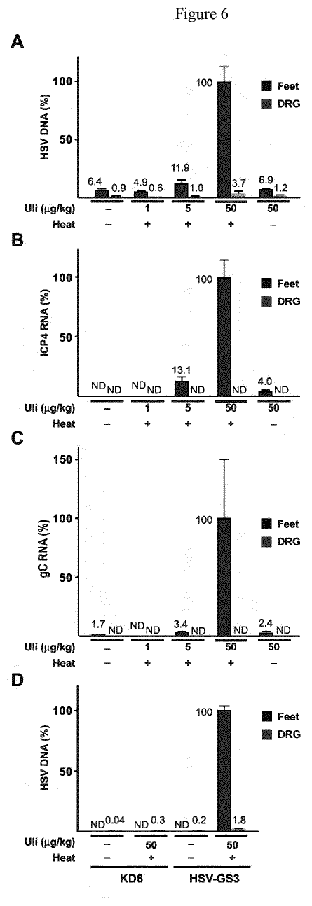

Results are shown in FIG. 6. FIG. 6A shows that replication in the feet depended both on heat treatment and small-molecule regulator. Furthermore, DNA replication was dependent on small-molecule regulator dose. Corresponding data were obtained for the expression of ICP4 genes (regulated genes) and the late gC gene (not subject to deliberate regulation) (FIGS. 6B & C). Small-molecule regulator alone (in the absence of a heat treatment) did essentially not stimulate DNA replication and at most marginally increased expression of ICP4 and gC transcripts.

Replicative yields of HSV-GS3 and replication-defective virus KD6 (ICP4-) were compared in the experiment shown in FIG. 6D. This experiment was performed as the previous experiment with the exception that mice were sacrificed 4 days post heat induction. Results showed that viral DNA could essentially only be detected in samples from the feet of HSV-GS3-infected animals that had received heat treatment and ulipristal. Very little DNA was found in DRG, and essentially none in KD-6-infected animals or in not-activated HSV-GS3-infected animals.

### Example 5: Immunization/Challenge Experiment Comparing a Replication-Competent Controlled Virus of the Invention with a Replication-Defective Comparison Virus (KD6)

The goal of this type of experiment was to demonstrate that immunizing mice with the HSV-GS3 virus under inducing conditions elicits a strong protective immune response against subsequent challenge with a lethal dose of wild-type HSV-1.

### (a) Survival

### Experimental Design:

TABLE 6
Immunization treatment groups

| | | | | HSV-GS3 |
|---|---|---|---|---|
| Group | Mock | KD6 | HSV-GS3 | (induced) |
| Immunization | vehicle | 50,000 pfu of | 50,000 pfu of | 50,000 pfu of |
| Tx* | (MEM + | KD6 (non- | HSV-GS3 | HSV-GS3 |
| | 10% FBS) | replicating, | | |
| | | ICP4-negative | | |
| | | HSV-1) | | |
| Challenge Tx | 10,000 pfu of | 10,000 pfu of | 10,000 pfu of | 10,000 pfu of |
| | HSV-1 strain | HSV-1 strain | HSV-1 strain | HSV-1 strain |
| | 17syn+ | 17syn+ | 17syn+ | 17syn+ |

*All Tx were in a volume of 0.050 ml/mouse.

a) Immunization: Mice were initially immunized in the experimental groups shown in Table 6. Each group contained 20 mice (ND4 Swiss Webster females, 4-6 weeks). Each vector was applied to the lightly abraded plantar surface of both rear feet following saline pre-treatment.
b) Induction: The HSV-GS3 vector was induced (or activated) in the “HSV-GS3 (induced)” group as follows: mifepristone (0.5 mg/kg) was administered i.p. at the time of immunization and again 24 h later. Heat was applied 3 h post immunization by immersing both rear feet in a 43.5° C. water bath for 30 min. Following immersion, the hind limbs were dried off, and the mice kept warm with a heat lamp until dry and warm.
c) Challenge: 22 days post immunization the mice were challenged with a 20-fold lethal dose of wild type HSV-1 strain 17syn+ applied to the lightly abraded plantar surface of both rear feet following saline pre-treatment. Efficacy of each immunization treatment was then assessed by a modified endpoint assay. Note that the modified endpoint assay involved euthanizing mice that were considered moribund (will not survive) based on clinical assessment (mice showing signs of bilateral hindlimb paralysis and CNS involvement, convulsions, and unable to move on their own to take food or water).

### Results:

Mice in the mock treatment group began to show signs of hindlimb paralysis and CNS infection as early as 6 days post challenge, while all three immunization groups appeared completely healthy until 8-9 days post challenge. Table 7 depicts the number of mice surviving at the end of the experiment (mice were followed 30 d post challenge). The results demonstrate that, while all treatments were able to protect at least some mice against a 20-fold lethal challenge of HSV-1, only the induced/activated HSV-GS3 virus treatment was able to afford substantial protection in the mice.

TABLE 7
Results of immunization/challenge experiment

| | No. of survivors (from groups |
|---|---|
| Group | of 20 animals) |
| HSV-GS3 Induced | 14 |
| KD6 | 5 |
| HSV-GS3 | 3 |
| Mock | 0 |

### (b) Replication of Challenge Virus

### Experimental Design:

a) Immunization: Mice were immunized as shown in Table 6. Each group contained 5 mice (ND4 Swiss females, 4-6 weeks). Each vector was applied to the lightly abraded plantar surface of the both rear feet following saline pre-treatment.
b) Induction: The HSV-GS3 vector was induced/activated in the “HSV-GS3 (induced)” group as follows: mifepristone (0.5 mg/kg) was administered i.p. at the time of immunization and again 24 h later. Heat was applied 3 h post immunization by immersing both rear hind feet in a 43.5° C. water bath for 30 min. Following immersion, the hindlimbs were dried off, and the mice kept warm with a heat lamp until dry and warm.
c) Challenge: 22 days post immunization the mice were challenged with a 20-fold lethal dose of wild type HSV-1 strain 17syn+ applied to the lightly abraded plantar surface of the both rear feet following saline pre-treatment. Four days post challenge the mice were euthanized and feet were dissected and homogenized. The tissue homogenates were then diluted and titrated on rabbit skin cells (RS) for infectious (17syn+) virus.

### Results:

Table 8 depicts the results of the titration data. These results illustrate that, while all of the immunization treatments were able to reduce replication to some extent (relative to mock) in the feet following challenge with HSV-1, HSV-GS3 (induced) mice showed by far the lowest challenge virus titer at four days post challenge (about two orders of magnitude lower than mock).

TABLE 8
Infectious virus (pfu) detected in the feet of mice, 4 d post challenge

| Mock | KD6 | HSV-GS3 | HSV-GS3 (induced) |
|---|---|---|---|
| 5.5 × 10⁵ +/− | 4.0 × 10⁴ +/− | 9.4 × 10⁴ +/− | 6.2 × 10³ +/− |
| 1.2 × 10⁴ | 8.3 × 10² | 3.1 × 10⁴ | 1.5 × 10² |

### Example 6: Immune Responses to an Influenza Hemagglutinin in the Context of Vigorous Viral Vector Replication

### Experimental Design:

a) Immunization: Mice were immunized as shown in Table 9. Each group contained 5 mice (6-8 week old female BALB/c mice). Each vector was applied to the lightly abraded plantar surface of the both rear feet following saline pre-treatment.
b) Induction: The HSV-GS3 and HSV-GS11 vectors were activated in the “HSV-GS3 (activated)”, “HSV-GS11 (activated)” and “HSV-GS11 (twice activated)” groups as follows: ulipristal (50 μg/kg) was administered i.p. at the time of immunization and again 24 h later. Heat was applied 3 h post immunization by immersing both rear hind feet in a 45° C. water bath for 10 min. Following immersion, the hindlimbs were dried off, and the mice were allowed to recover for 15 min at 37° C. Mice of the “HSV-GS11 (twice activated)” group were subjected to a second, identical heat and ulipristal (activation) treatment two days after the initiation of the first treatment.

TABLE 9
Immunization treatment groups

| | | | | | HSV- |
|---|---|---|---|---|---|
| | | | | | GS11 |
| | | HSV-GS3 | HSV- | HSV-GS11 | (twice |
| Group | Mock | (activated) | GS11 | (activated) | activated |
| Groups A | saline | 50,000 | 50,000 | 50,000 | 50,000 |
| for | | pfu of | pfu of | pfu of | pfu of |
| assessment | | HSV-GS3 | HSV- | HSV-GS11 | HSV- |
| of IEV HA | | | GS11 | | GS11 |
| expression | | | | | |
| Tx* | | | | | |
| Groups B | saline | 50,000 | 50,000 | 50,000 | 50,000 |
| for immune | | pfu of | pfu of | pfu of | pfu of |
| response | | HSV-GS3 | HSV- | HSV-GS11 | HSV- |
| assessment | | | GS11 | | GS11 |
| Tx | | | | | |

*All Tx (immunizations) were in a volume of 0.050 ml/mouse.

c) Further processing: One day after the last treatment, animals of groups A were euthanized, and RNA was extracted from one hindfoot and protein from the other.

Relative quantities of equine influenza (IEV) hemagglutinin (HA) RNA expression were assessed by reverse transcription and Taqman real-time PCR (RT-qPCR) using primers/probe disclosed in Table 3. (For methods, see also under Example 3.) Quantities of EIV HA were determined by an A/Equine/Prague/1/56 (EIV Prague/56) HA-specific ELISA: in order to produce the antigen for the ELISA, the insert from plasmid pBS-EIVPrague56/HA (that contains a cDNA sequence for the HA) was subcloned into expression vector pET-31b (Millipore) and EIVPrague56/HA protein was expressed in E. coli in accordance with the manufacturer's instructions. After induction and growth, the bacteria were lysed, proteins isolated in the presence of protease inhibitors, and the proteins used to coast a 96 well ELISA plate, and allowed to air-dry. Dilutions (1:10 to 1:200) of murine serum were applied to the wells and incubated at room temperature for 30 min. The serum was removed and the wells washed 2× with PBS. HRP conjugated anti-IgG antibodies were added to the wells, incubated for 30 min at room temperature and washed 2× with PBS. TMB substrate was added to the wells, and the plate was incubated for 10 minutes. The plate was then read with a spectrophotometer plate reader, and the results were evaluated based on the mean of sample to negative control mean (S/N) ratio.

Three weeks after the last treatment, serum was collected from all animals of groups B. For the collection of lymphocytes, the mice were anesthetized by inhalation of 2-3% isoflurane at 3 weeks after immunization. The total blood volume of each mouse was collected, and the mice were euthanized by cervical dislocation. PBMCs were isolated by Ficoll gradient separation using Lymphoprep (Miltenyi Biotec, Bergisch Gladbach. Germany) according to the manufacturer's protocol. To assess levels of neutralizing HA antibodies, serum samples were heated to 56° C. for 1 h to inactivate complement and were then diluted 1:10 in complete DMEM containing 10% heat-inactivated FBS. For EIV neutralization, 50 μl of a suspension containing approximately 100 TCID50 units of EIV Prague/56 were added to each dilution of serum to a final volume of 100 μl. Initial serum dilution therefore was 1:20. Serum-virus mixtures were placed on a rocker at room temperature for 1 h, and the amount of virus that was not neutralized at a given concentration of serum was titrated on MDCK cell monolayers in order to calculate the neutralizing antibody titers. EIV-specific T cells were quantified by a modified limiting dilution lymphoproliferation assay. Hayward A R, Zerbe G O, Levin M J. Clinical application of responder cell frequency estimates with four years follow up. J Immunol Methods. 1994; 170: 27-36. Briefly, wells of 96 well plates were coated with 20 μl/well of antigen extract (EIV Prague/56 HA or Vero cell control lysate) and were allowed to air-dry in a laminar flow hood. Dilutions of mouse peripheral blood mononuclear cells (PBMCs) in DMEM were added to each well such that each well contained a minimum of 1 and a maximum of 10 lymphocytes per well in a volume of 100 μl complete medium (with serum). The plates were then incubated at 37° C. After 24 h, medium containing 10 μCi of ³H-thymidine was added to each plate for 12 hours, the media replaced and the plates were incubated for an additional 72 h. The wells were harvested, the DNA precipitated in 20 volumes of cold 10% trichloroacetic acid, transferred onto glass fiber discs (Whatman GF/C) by filtration, rinsed with 95% ethanol, and dried using a heat lamp. The filters were then transferred to scintillation vials with Scintiverse (Fisher Scientific) and counted. The counts per minute (cpm) of ³H-thymidine were converted to RCF using the maximum likelihood estimate method of Levin et al. Levin M J, Oxman M N, Zhang J H, Johnson G R, Stanley H, Hayward A R, et al. Varicella-zoster virus-specific immune responses in elderly recipients of a herpes zoster vaccine. J Infect Dis. 2008; 197: 825-35.

### Results

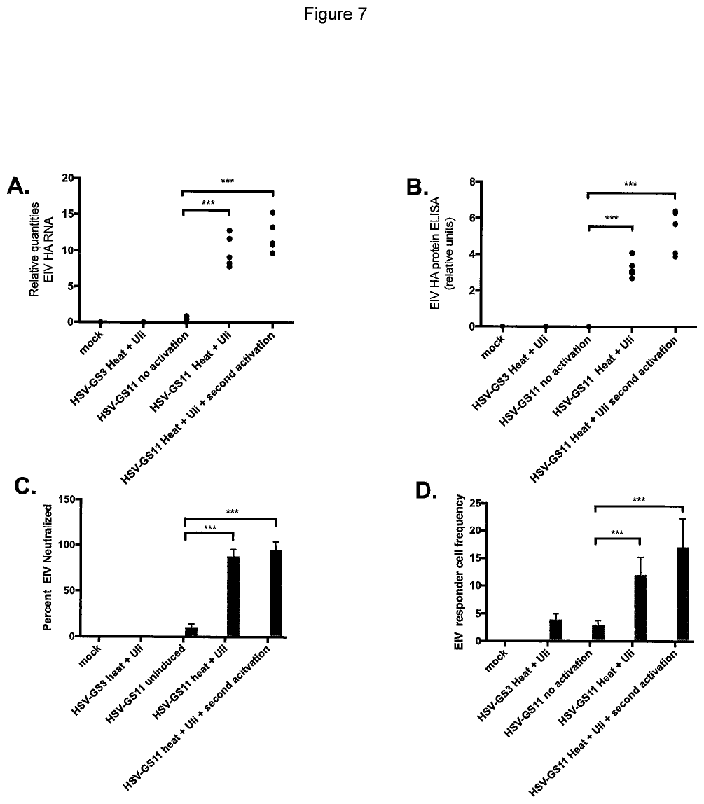

Results from an RT-qPCR analysis of HA gene expression are shown in FIG. 7A, and results from EIV Prague/56 HA-specific ELISA in FIG. 7B. HA RNA and HA could be detected in samples from HSV-GS11-inoculated animals, most abundantly when the animals had been subjected to activation treatment. Both HA RNA and protein levels appeared to be somewhat higher in twice-activated animals than in once-activated animals.

Immune responses were assessed three weeks after immunization. Serum samples were tested for their ability to neutralize EIV Prague/56. As expected, neutralizing antibodies were not detected in unimmunized (not shown), mock-immunized or vector-immunized animals (FIG. 7C). Unactivated HSV-GS11 was capable of inducing a neutralizing antibody response. Activation of HSV-GS11 shortly after inoculation resulted in a several-fold magnified response. It is noted that twice-activated HSV-GS11 elicited an only marginally better response than once-activated virus. HA-specific T cells present in PBMC were quantified by the above-described responder cell frequency assay. HA-specific T cells were not detected in unimmunized (not shown) or mock-immunized animals (FIG. 7D). Induction of a T cell response was observed in animals immunized with HSV-GS11 but not subjected to an activation treatment. Activated vector (HSV-GS3) produced a similar response. Far greater numbers of HA-specific T cells were found in animals immunized with once or twice activated HSV-GS11.

### Example 7: Activation of the HSPA7/HSP70B and the HSPA1A Promoter in Human Skin

A heating method that is inexpensive, operative anywhere in the field as well as applicable without the need for medical assistance may employ pads that are heated by the crystallization of a supercooled solution. This technology is well known (see U.S. Pat. No. 3,951,127 awarded to Watson and Watson in 1976) and has long been used in commercial articles such as, e.g., heating pads for soothing muscle or joint aches.

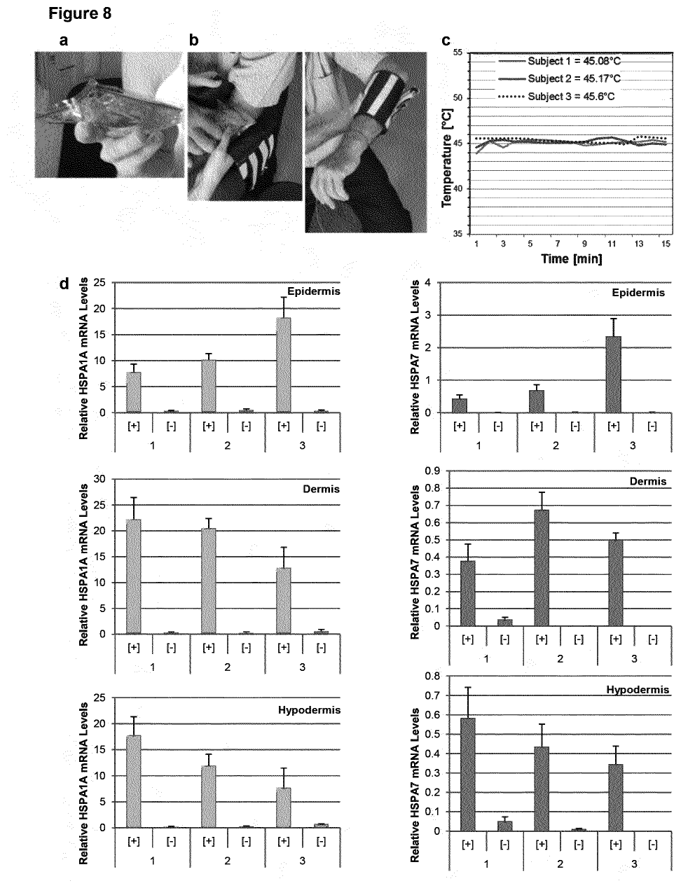

Proof-of-principle experiments aimed to deliver to human skin a heat dose near the upper end of the comfort zone but well below the threshold for skin damage. Moritz A, Henriques F (1947) Studies of thermal injuries II. The relative importance of time and surface temperature in the causation of thermal burns. Am J Pathol 23:695-720. A dose of about 45° C./15 min was considered appropriate. For the supercooled solution sodium thiosulfate pentahydrate was employed. This salt was chosen because it has a melting point of about 48° C., is inexpensive and essentially nontoxic. 10×10 cm heating pads were made from 0.1 mm thick PVC film (double-layered on the contact side) and contained 150 ml of sodium thiosulfate pentahydrate solution (99% pure; Fox Chemicals GmbH, Germany) that had been stabilized by the inclusion of 3% (weight) of distilled water (FIG. 8a). It was observed that the supercooled solution in the pad remained in the liquid state for several months when the pad was kept at room temperature. To ensure a tight contact between skin and heating pad, a water-based gel was applied to the area to be heated, here an area on an inner forearm of a subject. Crystallization was initiated, and the heating pad was placed on the forearm and was fastened using a sleeve having Velcro closures (FIG. 8b).

The data presented herein are from a self-experiment in which three principals of the study participated. The temperature evolution on the skin surface under the heating pad was measured by means of a calibrated thermocouple inserted between skin and heating pad. The intended operating temperature (45+/−0.5° C.) was reached 1-2 min after the heating pad had been affixed and was maintained within narrow limits throughout the remainder of the 15-min exposure period (FIG. 8c). Core body temperature did not change. Crystallization in the heating pad was reliably and rapidly triggered by a single prick with a fine needle. More elaborate starter mechanisms such as the inclusion in the pad of a snap metal disc or small rigid objects such as glass beads were described in U.S. Pat. Nos. 4,379,448 and 5,275,156. Thirty min after heat treatment, punch biopsies were taken from the center of the treated area as well as from a similar location on the contralateral arm. The cylindrical skin biopsies were embedded vertically in Tissue-Tek on metal holders, quick-frozen in liquid nitrogen and then stored at −80° C. Each sample was cut on a cryostat at a nominal section thickness of 40 μm. The texture of the sectioned tissue surfaces allowed differentiation between epidermis, dermis and hypodermis. The metal knife was carefully cleaned (70% ethanol) when moving from one tissue compartment to the next. Multiple sections (8-12) of each compartment were pooled, and RNA was extracted using a standard method. Extracted RNAs were analyzed by RT-qPCR for HSPA1A and HSPA7 transcripts, using β2-microglobulin transcripts for normalization. We found that the heat treatment resulted in strong activation of the HSPA1A and HSPA7 promoters in all three subjects. Relative HSPA1A transcript levels in heat-treated epidermal tissue were 7.75, 10.1 and 18.0 for subjects 1-3, and levels in untreated tissue were 0.39, 0.42 and 0.33, respectively (FIG. 8d, top graph). Fold induction was 19.7, 23.6 and 54.4, respectively. For HSPA7 transcripts, corresponding relative values were 0.43, 0.68 and 2.31 for heat-treated tissue and 0.0071, 0.0007 and 0.013 for untreated tissue. Induction was 60.0-, 944- and 171-fold. Similar findings were made for the other compartments, although lower fold induction values were also observed that were apparently due to elevated levels of uninduced expression in some tissue samples (FIG. 8d, middle and bottom graphs). It is noted that there was no obvious gradient of induced gene activity from epidermis to hypodermis. Comparable results were obtained when HSP transcript levels were normalized relative to RPS13 gene transcripts (not shown), except that normalized levels were depressed for one of the subjects who expressed considerably more RPS13 RNA than the other two. We conclude that the general method presented herein, which employs heating pads that heat by crystallization of sodium thiosulfate pentahydrate, was capable of effectively activating HSPA1A and HSPA7 promoters in all skin layers. This method can also be employed in the activation of a heat- and small-molecule regulator-controlled herpesvirus vector subsequent to its administration to a skin region.

### Example 8: Protective Immunization with Replication-Competent Controlled Virus HSV-GS11 Expressing an HA of A/Equine/Prague/1/56 (H7N7)

A mouse lethal challenge model was employed to demonstrate that immunization with activated HSV-GS11 induced a protective immune response. The choice of the foreign antigen expressed from HSV-GS11 was based on a paper by Kawaoka (J. Virol. (1991) 65: 3891-3894). The author had reported that equine H7N7 influenza viruses were lethal in BALB/c mice without mouse adaptation. Therefore, a hemagglutinin from an equine H7N7 virus, A/Equine/Prague/1/56 (H7N7) (EIV Prague/56), was selected as the foreign antigen. The design of the experiment is shown in Table 10.

TABLE 10
Immunization treatment groups

| Group | Group 1 | Group 2 | Group 3 | Group 4 |
|---|---|---|---|---|
| | Mock | HSV- | HSV-GS11 | HSV-GS11 |
| | | GS11 not | activated | activated |
| | | activated | | |
| Group size | 10 | 10 | 10 | 10 |
| First rear footpad | saline | 50,000 pfu | 50,000 | 50,000 |
| immunization | | HSV- | pfu HSV- | pfu HSV- |
| | | GS11 | GS11 | GS11 |
| Treatment* | No Tx | No Tx | Tx1 | Tx2 |
| Second rear footpad | saline | 50,000 pfu | 50,000 pfu | 50,000 pfu |
| immunization + Tx1 | | HSV- | HSV- | HSV- |
| or Tx2 | | GS11 | GS11 | GS11 |
| Challenge° | Prague/56 | Prague/56 | Prague/56 | Prague/56 |

HSV-GS11: Prague/56 HA;
°intranasal (<10^{b} EID₅₀) three weeks after second immunization.
*Treatment.
Tx1: 45° C./10 min heat to hind feet and 50 μg/kg ulipristal i.p.
Tx2: 44° C./10 min heat to hind feet and 50 μg/kg ulipristal i.p.
First and second immunizations used the same Tx.

Briefly, groups (n=10) of adult female BALB/c mice were immunized on the rear footpads with HSV-GS11 or saline as described in Examples 5 and 6. Ulipristal was administered intraperitoneally at the time of immunization. Three hours after virus administration, two of the four groups were subjected to heat treatment on their rear feet as described under Example 6. Note that two different heat treatment regimens were tested. After three weeks, the mice were reimmunized and virus reactivated. After another three weeks, all mice were challenged by intranasal administration of EIV Prague/56. The animals were then monitored daily for another three weeks and weights recorded. When mice reached clinical endpoints indicating severe influenza disease (>20% weight loss), they were euthanized. The results of the experiment revealed that immunization with activated HSV-GS11 protected against lethal influenza disease (100% protection for the more rigorous heat treatment, and 90% for the less rigorous treatment) (Table 11). No protection over mock immunization was observed in mice immunized with unactivated HSV-GS11.

TABLE 11
Survival data (percent animals surviving)

| Days after | Group | Group | Group | Group |
|---|---|---|---|---|
| challenge | 1 | 2 | 3 | 4 |
| 5 | 100 | 100 | 100 | 100 |
| 10 | 80 | 80 | 100 | 100 |
| 15 | 60 | 60 | 100 | 90 |
| 20 | 60 | 60 | 100 | 90 |

### Example 9: Protective Immunization with Replication-Competent Controlled Viruses HSV-GS10-13 Expressing Antigens from A/Equine/Prague/1/56 (H7N7) or A/Equine/Kentucky/1/94 (H3N8)

A/Equine/Prague/1/56 (H7N7) is abbreviated (EIV) Prague/56 below and A/Equine/Kentucky/1/94 (H3N8) is (EIV) Kentucky/94.

The experiment employed the same model and procedures that were described in Example 8. The design of the experiment is shown in Table 12.

TABLE 12
Immunization treatment groups

| Group | Group 1 | Group 2 | Group 3 | Group 4 |
|---|---|---|---|---|
| | Mock | HSV- | HSV- | HSV-GS3 |
| | | GS10 & −11 | GS12 & −13 | (activated) |
| | | (activated) | (activated) | |
| Group size | 10 | 10 | 10 | 10 |
| First rear footpad | saline | 250,000 | 250,000 | 250,000 |
| immunization | | (2.5 × 10⁵) | (2.5 × 10⁵) | (2.5 × 10⁵) |
| | | pfu each | pfu each | pfu |
| | | of GS10 & | of GS12 & | HSV-GS3 |
| | | GS11 | GS13 | |
| Treatment* | No Tx | Tx | Tx | Tx |
| Second rear | saline | 250,000 | 250,000 | 250,000 |
| footpad | | (2.5 × 10⁵) | (2.5 × 10⁵) | (2.5 × 10⁵) |
| immunization + | | pfu each | pfu each | pfu |
| Tx | | of GS10 & | of GS12 & | HSV-GS3 |
| | | GS11 | GS13 | |
| Challenge° | Prague/56 | Prague/56 | Prague/56 | Prague/56 |

HSV-GS10: Prague/56 nucleoprotein;
HSV-GS11: Prague/56 HA;
HSV-GS12: Kentucky/94 nucleoprotein;
HSV-GS13: Kentucky/94 HA;
°intranasal (>10⁶ EID₅₀) three weeks after second immunization.
*Treatment.
Tx: 44.5° C./10 min heat to hind feet and 50 μg/kg ulipristal i.p.
First and second immunizations used the same Tx.

Results of the experiments are shown in Table 13 below.

TABLE 13
Survival data (percent animals surviving)

| Days after | Group | Group | Group | Group |
|---|---|---|---|---|
| challenge | 1 | 2 | 3 | 4 |
| 5 | 80 | 100 | 100 | 70 |
| 10 | 0 | 100 | 60 | 0 |
| 15 | 0 | 100 | 60 | 0 |
| 20 | 0 | 100 | 60 | 0 |

The results of the experiment revealed that immunization with a combination of activated HSV-GS10 and HSV-GS11 expressing antigens from Prague/56 protected fully against a lethal challenge with Prague/56 virus. Highly significant cross-protective immunity was induced in animals that had been immunized with a combination of activated HSV-GS12 and HSV-GS13 expressing antigens from Kentucky/94. Sixty percent of animal survived a lethal challenge by the heterosubtypic Prague/56 virus. HSV-GS10-13 had been derived from HSV-GS3 (that does not express an influenza antigen). No protective immunity was induced by HSV-GS3 or mock immunization.

All references cited in this application, including publications, patents and patent applications, shall be considered as having been incorporated in their entirety.

1-11. (canceled)

12. A method of immunization of a mammalian subject against an influenza virus strain, the method of immunization comprising (a) administering to an inoculation site region in the body of the mammalian subject a composition comprising an effective amount of a replication-competent controlled herpesvirus which is a recombinant virus in which one or more replication-essential genes have been placed under the control of a gene switch that is inserted in the genome of the recombinant virus and that can be activated deliberately, and (b) exposing the inoculation site region of the mammalian subject to a localized activation treatment that activates the recombinant virus to undergo a round of replication in the inoculation site region, wherein the replication-competent controlled herpesvirus carries an expressible gene for an antigen of an influenza virus strain that is different from the influenza virus strain against which the immunization is directed.

13. The method of immunization according to claim 12, wherein the activation treatment comprises administering an activating heat dose to the inoculation site region.

14. The method of immunization according to claim 13, wherein the replication-competent controlled herpesvirus is a recombinant herpesvirus that comprises an inserted gene encoding a small-molecule regulator-activated transactivator which gene is functionally linked to a nucleic acid sequence that acts as a heat shock promoter as well as a transactivator-responsive promoter, and one or more transactivator-responsive promoters that are functionally linked to the one or more replication-essential genes.

15. The method for immunization according to claim 14, wherein the replication-competent controlled herpesvirus is a recombinant HSV-1 or HSV-2 and the replication-essential viral genes that are functionally linked to transactivator-responsive promoters include at least all copies of the ICP4 gene or the ICP8 gene.

16. The method for immunization according to claim 14, wherein the small-molecule regulator-activated transactivator contains a truncated ligand-binding domain from a progesterone receptor and is activated by a progesterone receptor antagonist that is capable of interacting with the ligand-binding domain and activating the transactivator.

17. The method of immunization according to claim 13, wherein the replication-competent controlled herpesvirus is a recombinant virus selected from the group consisting of an HSV-1, an HSV-2, a varicella zoster virus and a cytomegalovirus.

18. The method of immunization according to claim 13, wherein the replication-competent controlled herpesvirus is a recombinant herpesvirus that comprises an inserted gene encoding a transactivator activated by a small-molecule regulator, wherein the gene encoding the transactivator is functionally linked to a nucleic acid that acts as a heat shock promoter, and one or more transactivator-responsive promoters that are functionally linked to one or more replication-essential genes.

19. The method of immunization according to claim 13, wherein the replication-competent controlled herpesvirus is a recombinant herpesvirus that comprises an inserted gene encoding a small-molecule regulator-activated transactivator wherein the gene encoding the transactivator is functionally linked to a nucleic acid that acts as a constitutively active promoter or a transactivator-responsive promoter, a first replication-essential gene of the replication-competent controlled herpesvirus that is functionally linked to a promoter activated by heat and a second replication-essential gene of the replication-competent controlled herpesvirus that is functionally linked to a transactivator-responsive promoter.

20. The method of immunization according to claim 13, wherein the replication-competent controlled herpesvirus carries an expressible gene for an antigen of an influenza virus strain that differs in clade from the influenza virus strain against which the immunization is directed.

21. The method of immunization according to claim 13, wherein the replication-competent controlled herpesvirus carries an expressible gene for an antigen of an influenza virus strain that differs in subtype from the influenza virus strain against which the immunization is directed.

22. The method of immunization according to claim 13, wherein the inoculation site region is a cutaneous or subcutaneous region on the trunk or on an extremity of the mammalian subject.

23. The method of immunization according to claim 13, wherein the expressible gene for an antigen of an influenza virus strain is a gene or gene fragment encoding all or part of a nucleoprotein, a hemagglutinin, a neuraminidase, an ion channel protein or a matrix protein.
